(19) United States
(12) Patent Application Publication
(10) Pub. No.: US 2015/0123670 A1
(43) Pub. Date: May 7, 2015

(54) IN SITU SENSING OF COMPOUNDS

(71) Applicant: Tufts University, Medford, MA (US)

(72) Inventors: Albert Robbat, JR., Andover, MA (US); Patrick Antle, Cambridge, MA (US)

(73) Assignee: Tufts University, Medford, MA (US)

(21) Appl. No.: 14/347,541

(22) PCT Filed: Sep. 26, 2012
(86) PCT No.: PCT/US12/57340

§ 371 Date: Mar. 26, 2014

Related U.S. Application Data
(60) Provisional application No. 61/539,698, filed on Sep. 27, 2011.

(51) Int. Cl. G01N 27/70; G01N 33/00

(52) U.S. Cl. CPC G01N 27/70; G01N 33/0027
USPC 324/464

(57) ABSTRACT

The disclosure features systems and methods for detecting organic compounds that include: (a) a transfer line; (b) a probe connected to a first end of the transfer line, the probe including an inlet port and a membrane positioned across an opening in the inlet port; (c) an analysis unit connected to a second end of the transfer line; and (d) an electronic controller. The analysis unit can include a valve featuring multiple ports, a first detection unit configured to measure a photoionization current, and a second detection unit configured to identify chemical compounds, and a trap configured to condense chemical compounds from a vapor phase to a liquid phase. The electronic controller is connected to the valve, the first and second detection units, and the trap.

### CROSS-REFERENCE TO RELATED APPLICATIONS

This application claims priority under 35 U.S.C. §119 to U.S. Provisional Patent Application No. 61/539,698, filed on Sep. 27, 2011, the entire contents of which are incorporated herein by reference.

### STATEMENT AS TO FEDERALLY SPONSORED RESEARCH

This invention was made with Government support under EPA grant number EP-D-10-062. The United States Government has certain rights in this invention.

### TECHNICAL FIELD

This disclosure relates to in-situ sensing, characterization, and remote analysis of compounds, such as those found at contaminated environmental sites or buildings, and process monitoring where the sample is brought to a detector from a distance, such as a distance of three feet or more.

### BACKGROUND

The characterization of sub-surface pollutants at contaminated sites presents a number of challenging problems. Pollutants present at such sites can be analyzed by gas chromatography-mass spectrometry (GC-MS) after the sample is brought to the surface. However, the heterogeneity and compositional variability even at a single site makes development of generally applicable systems and methods difficult. Further, because large site areas are surveyed frequently, methods and systems used for such characterization should provide accurate results in a relatively short time. Conventional methods for sensing of pollutants such as organic compounds using gas chromatography and/or mass spectrometry are typically limited by the requirement that the compounds remain in the gas phase throughout.

### SUMMARY

This disclosure features traps for collecting and condensing organic compounds. The traps are compact, capable of condensing compounds at temperatures of −30° C. or lower, and can be both cooled and heated using devices such as fans and Peltier cooling elements. The disclosure also features flow regulators that include a valve with multiple ports, and a sample injector connected to at least one of the multiple ports. Compounds can be introduced into the flow regulator through more than one port and in a variety of different physical states, and the configuration of the valve can be adjusted to direct compounds along multiple flow paths leading to traps and/or detectors. The disclosure further features membranes that include fluorinated coating materials applied to a supporting material to impart water-excluding capability. The membranes can be used at temperatures of 300° C. or more.

In general, the traps, flow regulators, and membranes disclosed herein can each be used in a variety of different applications and systems. Combinations of one, two, or more, or all, of these components can also be used in various systems. This disclosure features methods and systems for detecting compounds, and the systems can include any one, any two, or all of the components described herein, in any combination. The methods and systems permit in situ detection of compounds in a wide variety of environments, and under various conditions. In some embodiments, for example, a probe at the end of an extended transfer line separates compounds for analysis from materials to which they are adsorbed, and conveys the compounds to an online GC-MS analysis unit. The probe can include a fluorinated polymer membrane that permits the probe to be used in environments where the water concentration is relatively high by substantially preventing water from entering a transfer line that conveys compounds from the site of recovery to the GC-MS unit. Compounds that are separated (e.g., by thermal desorption) from supporting material are transferred through a specially-designed multi-way inlet valve and into three-stage, Peltier-based freeze trap that condenses the compounds prior to injection into the GC-MS unit. By using the multi-way inlet valve and the freeze trap, both volatile organic compounds (VOCs) and semi-volatile organic compounds (SVOCs) are condensed in the trap at the same time, permitting simultaneous analysis of these classes of compounds in the GC-MS unit.

In general, in a first aspect, the disclosure features traps for condensing organic compounds that include a condenser coil forming a flow path for gas, a cooling element, a first set of electrical contacts connected to the condenser coil, and a second set of electrical contacts connected to the cooling element, where during operation the trap is configured to be heated by directing an electrical current to pass through the condenser coil, and configured to be cooled by directing an electrical current to pass through the cooling element.

Various implementations of the traps can include any one or more of the following features.

The condenser coil can include stainless steel. The cooling element can include a Peltier cooling chip (e.g., a stack of three Peltier cooling chips). The trap can include a second cooling element, where the condenser coil is positioned between the two cooling elements. One of the cooling elements can be a passive cooling element with no moving components, and the other cooling element can be an active cooling element with one or more moving components. The active cooling element can include a fan.

During operation, the cooling element can be configured to maintain the condenser coil at a temperature of −30° C. or less.

A condenser system can include the trap and an electronic processor, which in one configuration is connected to the second set of electrical contacts and configured to condense organic compounds in the condenser coil by directing an electrical current to pass through the cooling element to reduce a temperature of the condenser coil. In another configuration, the electronic processor can be connected to the first set of electrical contacts and configured to vaporize condensed organic compounds in the condenser coil by directing an electrical current to pass through the condenser coil to increase a temperature of the condenser coil. The electronic processor also can be configured to concentrate organic compounds in the trap by condensing organic compounds in the condenser coil during a first time interval, and then vaporizing the condensed organic compounds during a second time interval, where the first time interval is larger or longer than the second time interval by a factor of 2.5 or more.

Various implementations of the traps can also include any of the other features disclosed herein, in any combination, as appropriate.

In another aspect, the disclosure features flow regulators that include a valve that includes at least six ports and defines at least three flow paths among the ports, a sample injector connected to a first one of the ports, and a detector connected to the sample injector, where in a first configuration, the flow regulator is configured so that gas molecules enter the valve through a second one of the ports, enter the sample injector through the first port, and are detected by the detector, and in a second configuration, the flow regulator is configured so that gas molecules enter the valve through the second port, and enter a trap connected to a third one of the ports.

Various implementations of the flow regulators can include any one or more of the following features.

The detector can be a photoionization detector. The second port can be configured to connect to a transfer line, where gas molecules in the transfer line enter the valve through the second port. The sample injector can include an aperture configured to admit a syringe, and in the second configuration, a sample introduced through the aperture can enter the valve through the first port and can enter a trap connected to a fourth one of the ports. The flow regulator can be configured so that a common trap is connected to the third and fourth ports.

The detector can be detachably connected to the sample injector, and the flow regulator can be configured to receive a sample adsorbed onto an adsorbent material through the sample injector when the detector is disconnected from the sample injector. In the second configuration, a sample received through the sample injector when the detector is disconnected can enter the valve through the first port and can enter a trap connected to a fourth one of the ports.

The sample injector can be a first sample injector, and the flow regulator can include a second sample injector connected to a fifth one of the ports, where in the first configuration, a sample introduced through the second sample injector enters the valve through the fifth port and enters the trap connected to the fourth port. The flow regulator can be configured so that a common trap is connected to the third and fourth ports.

A condenser system can include the flow regulator, and an electronic processor connected to the flow regulator and the detector, where the electronic processor is configured to maintain the flow regulator in the first configuration until gas molecules are detected by the detector, and to change the flow regulator to the second configuration when gas molecules are detected by the detector. The electronic processor can be configured to reduce a temperature of the trap when the flow regulator is in the second configuration to condense the gas molecules in the trap. The electronic processor can be configured to return the flow regulator to the first configuration after a period of time in the second configuration, where in the first configuration, the gas molecules enter the valve through the third port and enter an analysis unit connected to a sixth one of the ports. The electronic processor can be configured to increase a temperature of the trap when the flow regulator is returned to the first configuration. A direction of gas flow in the trap can reverse when the flow regulator is returned to the first configuration from the second configuration.

Embodiments of the flow regulators can also include any of the other features disclosed herein, in any combination, as appropriate.

In a further aspect, the disclosure features water-excluding membranes that include a supporting material featuring a plurality of openings, and a fluorinated coating material applied to the supporting material, where the membrane does not allow water to pass through at a temperature of up to or at 300° C.

Various implementations of the membranes can include any one or more of the following features.

The supporting material can include a stainless steel mesh. The plurality of openings can have an average maximum dimension of between 20 microns and 200 microns.

The fluorinated coating material can include a polytetrafluoroethylene material. The membrane may not allow water to pass through at a temperature of up to or at 400° C. Pores in the membrane can be sized to permit organic molecules to pass through the membrane. The membrane can be impermeable to liquid water and to steam.

Various implementations of the membranes can also include any of the other features disclosed herein, in any combination, as appropriate.

In another aspect, the disclosure features methods of fabricating a water-excluding membrane that includes applying a layer of a fluorinated coating material to a first surface of a supporting material, heating the supporting material to a temperature of at least 300° C. for a first period of at least 30 minutes, applying a layer of the fluorinated coating material to a second surface of the supporting material, and heating the supporting material to a temperature of at least 300° C. for a second period of at least 30 minutes.

Embodiments of the methods can include any one or more of the following features.

Applying the layer of the fluorinated coating material to the first surface can include applying the coating material dropwise to the first surface. Applying the layer of the fluorinated coating material to the first surface can include brushing the applied coating material on the first surface. Applying the layer of the fluorinated coating material to the second surface can include spraying the coating material onto the second surface.

The methods can include, after applying the layer of the fluorinated coating material to the first surface and before heating the supporting material for the first period, heating the supporting material to a temperature of at least 100° C. for a third period, and heating the supporting material to a temperature of at least 200° C. for a fourth period. The methods can include, after applying the layer of the fluorinated coating material to the second surface and before heating the supporting material for the second period, heating the supporting material to a temperature of at least 100° C. for a fifth period, and heating the supporting material to a temperature of at least 200° C. for a sixth period. The methods can include repeating the steps of applying a layer of the fluorinated coating material to the second surface, heating the supporting material to a temperature of at least 100° C. for a seventh period, heating the supporting material to a temperature of at least 200° C. for an eighth period, and heating the supporting material to a temperature of at least 300° C. for a ninth period. The steps can be repeated so that three layers of the fluorinated coating material are applied to the second surface. The methods can include heating the supporting material to a temperature of at least 300° C. for a tenth period of at least one hour.

A thickness of the layer of fluorinated coating material applied to each of the first and second surfaces can be 50 microns or less. The fluorinated coating material can include a polytetrafluoroethylene material. The supporting material can include a stainless steel mesh.

Embodiments of the methods can also include any of the other features or steps disclosed herein, in any combination, as appropriate.

In a further aspect, the disclosure features systems for detecting organic compounds that include: (a) a transfer line; (b) a probe connected to a first end of the transfer line, the probe including an inlet port and a membrane positioned across an opening in the inlet port; (c) an analysis unit connected to a second end of the transfer line and featuring a valve including multiple ports where a first one of the multiple ports is connected to the transfer line, a first detection unit configured to measure a photoionization current for chemical compounds and connected to a second one of the multiple ports, a second detection unit configured to identify chemical compounds and connected to a third one of the multiple ports, and a trap configured to condense chemical compounds from a vapor phase to a liquid phase and connected to a fourth and a fifth ones of the multiple ports; and (d) an electronic controller connected to the valve, the first and second detection units, and the trap.

Various implementations of the systems can include any one or more of the following features.

The membrane can include a fluorinated coating material applied to a supporting material, where the membrane does not undergo degradation when heated to a temperature of 300° C. A thickness of the fluorinated coating material can be 50 microns or less. The membrane can be impermeable to water and steam, and the membrane can be permeable to at least some organic molecules.

The systems can include a first sample injector connected between the second port and the first detection unit. The first sample injector can include an aperture configured to admit a syringe. The system can include a second sample injector connected to a sixth one of the multiple ports, the second sample injector including an aperture configured to admit a syringe.

The traps can include a condenser coil forming a flow path for gas and a first set of electrical contacts connected to the condenser coil, and a cooling element adjacent to the condenser coil and a second set of electrical contacts connected to the cooling element. The cooling element can include a stack of three Peltier cooling chips. During operation, the cooling element can be configured to maintain the condenser coil at a temperature of −30° C. or less.

The electronic controller can be configured to reduce a temperature of the condenser coil by applying an electrical signal to the second set of electrical contacts, and to increase the temperature of the condenser coil by applying an electrical signal to the first set of electrical contacts. The probe can include a heating element connected to the electronic controller, and during operation the electronic controller can be configured to apply an electrical signal to the heating element to maintain the probe at a temperature of 300° C. or more.

The valves can include a first configuration that defines a first flow path between the first and second ports in the valve, and during operation, the electronic controller can be configured to adjust the analysis unit so that the valve is in the first configuration and molecules in the transfer line enter the first port and are detected by the first detector. The valve can include a second configuration that defines a second flow path between the first and fifth ports in the valve, and during operation, when molecules are detected by the first detector, the electronic controller is configured to adjust the analysis unit so that the valve is in the second configuration and molecules from the transfer line are condensed in the trap. The electronic controller can be configured to reduce a temperature of the trap to condense the molecules.

The first configuration can define a third flow path between the third and fifth ports, and during operation, when the molecules have been condensed in the trap, the electronic controller can be configured to adjust the analysis unit so that the valve is in the first configuration and the condensed molecules are detected by the second detector. The electronic controller can be configured to increase a temperature of the trap to vaporize the condensed molecules. Gas flow through the trap can occur in a first direction when the valve is in the first configuration, and in a second direction opposite to the first direction when the valve is in the second configuration.

A length of the transfer line can be 3 meters or more.

Various implementations of the systems can also include any of the other features disclosed herein, in any combination, as appropriate.

In another aspect, the disclosure features methods for detecting organic compounds that include: (a) directing molecules of one or more organic compounds to flow through a transfer line and to enter a valve through a first one of multiple valve ports, where the valve includes a first configuration defining a flow path between the first port and a second one of the multiple ports; (b) detecting molecules from the transfer line with a first detector connected to the second port; (c) adjusting the valve to a second configuration defining a flow path between the first port and a third one of the multiple ports; (d) condensing molecules from the transfer line in a trap connected to the third port; (e) adjusting the valve to the first configuration, where the first configuration defines a flow path between the third port and a fourth one of the multiple ports; (f) vaporizing the condensed molecules; and (g) detecting the vaporized molecules with a second detector connected to the fourth port.

Embodiments of the methods can include any one or more of the following features.

A length of the transfer line can be 3 meters or more.

The method can include reducing a temperature of the trap to condense the molecules in the trap. The method can include increasing a temperature of the trap to vaporize the condensed molecules.

Detecting molecules with the first detector can include measuring a photoionization current associated with the molecules. Detecting molecules with the second detector can include detecting the molecules using at least one of a gas chromatography detector and a mass spectrometry detector.

A first end of the transfer line can be positioned below a ground surface, and directing molecules of one or more organic compounds to flow through the transfer line can include thermally desorbing the molecules from a soil matrix material adjacent to the first end of the transfer line. Thermally desorbing the molecules can include heating the soil matrix material to a temperature of 300° C. or more.

The one or more organic compounds can include at least one volatile organic compound (VOC) and at least one semi-volatile organic compound (SVOC), and the at least one VOC and the at least one SVOC can be thermally desorbed at a common temperature from the soil matrix material.

Directing molecules of one or more organic compounds to flow through the transfer line can include directing the molecules to pass through a membrane that includes a fluorinated coating material that does not degrade at a temperature of 300° C. The soil matrix material can have a water concentration of 15% or more by weight.

Adjusting the valve from the first to the second configuration or from the second to the first configuration can reverse a direction of gas flow in the trap.

Embodiments of the methods can also include any of the other features or steps disclosed herein, in any combination, as appropriate.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features and advantages of the invention will be apparent from the description, drawings, and claims.

### DESCRIPTION OF DRAWINGS

FIG. 1 is a schematic diagram of a system for in situ analysis of sub-surface, e.g., underground compounds.

FIG. 2 is a representation of a truck configured to transport the system of FIG. 1.

FIG. 3 is a representation of a cone penetrometer assembly.

FIG. 4A is a schematic diagram of a transfer line.

FIG. 4B is a schematic cross-sectional view of the transfer line of FIG. 4A.

FIG. 5 is a photograph showing an inlet probe.

FIG. 6A is a schematic diagram showing an inlet probe.

FIG. 6B is a schematic exploded view of the inlet probe of FIG. 6A.

FIG. 6C is a schematic view of a heating block and collection port of the inlet probe of FIG. 6A.

FIG. 7 is a schematic diagram showing a multi-way valve.

FIGS. 8A-8C are schematic diagrams showing different port configurations of the multi-way valve of FIG. 7.

FIG. 9A is a schematic view of an inlet probe, multi-way valve, and freeze-trap assembly.

FIG. 9B is a schematic exploded view of a portion of the assembly of FIG. 9A.

FIG. 9C is a schematic exploded view of a calibration unit.

FIG. 10A is a schematic view of a low temperature freeze trap and Peltier cooler assembly.

FIG. 10B is a schematic exploded view of the assembly of FIG. 10A.

FIG. 11 is a plot showing total ion current (TIC) and reconstructed ion current (RIC) chromatograms for field tests of the system of FIG. 1.

FIG. 12 is a plot showing total ion current (TIC) and reconstructed ion current (RIC) chromatograms for laboratory tests of the system of FIG. 1.

Like reference symbols in the various drawings indicate like elements.

### DETAILED DESCRIPTION

This disclosure features systems and methods for in situ detection and analysis of compounds in diverse environments. An important application for these new systems and methods is sub-surface detection of organic pollutants at contaminated industrial and military sites. Although certain tools exist for hazardous waste characterization at such sites, it has generally been difficult to detect and analyze the wide variety of different organic pollutants that forensic investigators need to target. For example, direct measuring thermal extraction mass spectrometry has been used to detect volatile organic compounds (VOCs) successfully, but detection of semi-volatile organic compounds (SVOCs) using the same instruments and techniques has proven to be challenging. On-line detection of SVOCs continues to present challenges because of difficulties associated with efficient extraction, transfer, and quantitation of all targeted analytes, because saturation of mass spectrometry (MS) detectors leads to long bakeout periods for recovery, and because standard Environmental Protection Agency (EPA) methods separate analysis of VOCs and SVOCs so that field personnel typically wait for analysis of one class of compounds to be completed before analysis of the other class begins.

The systems and methods disclosed herein are discussed in the context of application to the analysis and detection of organic pollutants at contaminated industrial and military sites as part of forensic site characterization. It should be noted at the outset, however, that other applications exist for the systems and methods; and certain additional applications will be discussed later.

The systems disclosed herein include an analysis unit such as a gas chromatography-mass spectrometry (GC-MS) instrument coupled to a resistively-heated transfer line. The transfer line is threaded through a collection member (e.g., a pipe that extends below the soil surface to a depth of up to about 100 meters). A probe connected to the end of the transfer line separates compounds for analysis from confounding background matrix materials, and allows the separated and volatilized organic compounds to be transported through the transfer line to the analysis unit. This disclosure is divided into multiple sections. The first section discloses general methodologies and systems for performing in situ detection and analysis of contaminants. Subsequent sections discuss various additional aspects and features of the systems and methods.

### General Systems and Methodologies

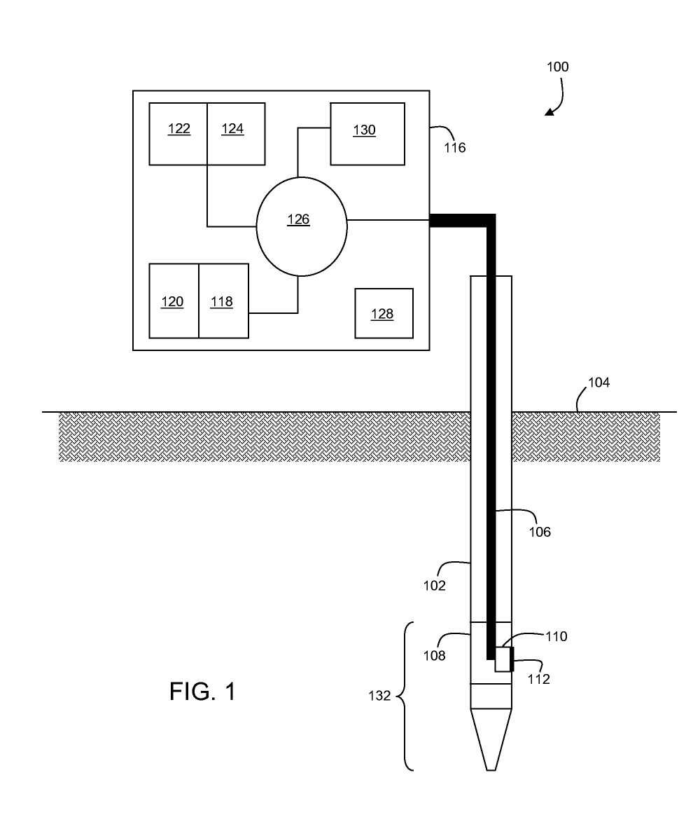

FIG. 1 shows a schematic diagram of a system 100 configured to perform sub-surface detection of pollutants (e.g., organic pollutants) at a contaminated site. System 100 includes a pipe 102 that extends below the surface 104 of soil at the site. Positioned within pipe 102 is a transfer line 106 that extends through the top of pipe 102 and above surface 104. A second pipe 108 is connected to pipe 102 and houses sample collection port 110. Transfer line 106 is in fluid communication with second pipe 108; the fluid connection extends to sample collection port 110. Positioned in front of sample collection port 110 to prevent matrix material and water from entering transfer line 106 is inlet membrane 112. Together, second pipe 108, sample collection port 110, and inlet membrane 112 form inlet probe 132.

The other end of transfer line 106 is coupled to analysis unit 116. Analysis unit 116 includes a gas chromatography (GC) unit 118 and a coupled mass spectrometry (MS) unit 120. Analysis unit 116 also includes a photoionization detector (PID) 130. Compounds collected from the soil below surface 104 are conveyed through transfer line 106 into analysis unit 116. The compounds are heated when they enter transfer line 106 to cause thermal desorption from soil matrix materials to which they are ordinarily bound, and mixed with a carrier gas such as nitrogen so that they propagate through transfer line 106 and enter analysis unit 116 in the vapor phase. Once inside analysis unit, the compounds are first condensed onto an inert material in freeze trap 122, and then thermally desorbed from the inert material in desorber unit 124 (which can be integrated into freeze trap 122 in some embodiments). Inert gas (e.g., nitrogen) is introduced via multi-way inlet valve 126, and it is used to sweep the desorbed organic compounds into the GC and MS units 118 and 120, in succession, for analysis.

A processing unit 128 is in electrical communication with transfer line 106, GC unit 118, MS unit 120, freeze trap 122, desorber unit 124, and multi-way valve 126. Typically, processing unit 128 includes one or more electronic processors, a data storage medium, a memory unit, a communications interface, a display unit, and a human interface device. Processing unit 128 can be configured to monitor and/or control a variety of different system parameters. For example, processing unit 128 can control temperature (e.g., the temperature of sample collection port 110, the temperature of transfer line 106, the temperature of desorber unit 124, and/or the temperature of freeze trap 122). To control the temperature of transfer line 106, for example, processing unit 128 can direct an electrical current to pass through transfer line 106, resistively heating the transfer line.

Processing unit 128 can also control flow rates (e.g., the flow rate of carrier gas through transfer line 106 and/or the flow rate of gases through freeze trap 122, desorber unit 124, GC unit 118, and MS unit 120). Processing unit 128 can also be configured to control gas pressures in any of the system components described herein. Further, processing unit 128 can be configured to monitor temperatures, pressures, flow rates, and other parameters in the various components of the system.

The data storage medium can include a variety of different devices for storing data, and can include both on-board storage media and remote storage media (e.g., storage media connected to processing unit 128 via a wired or wireless network, such as an intranet or the Internet). In some embodiments, the data storage medium is a magnetic and/or optical storage medium. In certain embodiments, the data storage medium includes a data logger and/or a printer. In some embodiments, the data storage medium is connected to the other components of processing unit 128 via a data network, but is located remotely. Data recorded by system 100 and/or analysis results can be transmitted to the data storage device via the network. In certain embodiments, information received by a data storage medium over a network can be further processed by one or more additional processors connected to the data storage medium. For example, a computer or handheld computing device (e.g., a mobile phone) can be used to further process, store, or display information received from system 100, and can also issue commands to system 100 over the network.

In general, data and analysis results can be transmitted via a variety of different types of connections to storage media and other processors located remotely. System operators located remotely can receive the data and analysis results, which can then be subjected to further analysis. As an example, the results of analysis of organic contaminants at a particular location can be transmitted over a network to a remote office for use in construction of site visualization maps for the location.

Pipe 102 can be formed either as a single continuous pipe member, or as a plurality of pipe sections, with transfer line 106 threaded through each of the sections. Pipe 102 can be formed of a variety of materials. In some embodiments, for example, pipe 102 is formed of relatively rigid materials such as steel. In certain embodiments, pipe 102 can be formed of materials that are more flexible than steel, but that can still function as a flexible sheath to protect transfer line 106. In some embodiments, pipe 102 is formed from the same material as one or more of the materials used to form cooling loops in freeze traps (which will be discussed subsequently).

In certain embodiments, system 100 can be used without pipe 102. That is, transfer line 106—without a surrounding pipe member—is used to collect samples. In the absence of a pipe member, transfer line 106 can, in some embodiments, be surrounded by one or more protective layers (e.g., one or more layers of cloth tape and/or a rubber or plastic sheath). Alternatively, transfer line 106 can be used without any additional layers applied to its surface. Transfer line 106 can be formed from a variety of materials, including materials such as rubber and/or one or more hydrocarbon polymers (e.g., synthetic rubber and fluoropolymer elastomers, such as Viton®), as described in further detail below. Certain applications are particularly well suited to the use of transfer line 106 without a rigid pipe 102 (e.g., without any protective covering, or with one or more protective layers, or a flexible pipe 102 that functions as a protective sheath). For example, when system 100 is used to detect mold and other contaminants indoors (e.g., in ducts, inside walls, between floors, and in other crevices), it can be advantageous if transfer line 106 is highly flexible to be able to collect samples from locations that would otherwise be inaccessible.

In some embodiments, pipe 102 can be formed as a single unitary member. In certain embodiments, pipe 102 can be assembled from a plurality of sections. The lengths of the sections can be selected for ease of assembly and/or cost. For example, pipe 102 can be assembled from a plurality of sections, each of length between 0.2 m and 4.0 m (e.g., between 0.4 m and 3.0 m, between 0.6 m and 2.0 m).

The outside diameter of pipe 102 can be selected according to the sizes of the components housed within the pipe, and according to the environment in which system 100 (and in particular, pipe 102) is deployed. For example, when relatively large forces are applied to pipe 102 to force it to penetrate below the surface of soil, the outside and inside diameters of the pipe can be selected to yield a pipe with relatively thick walls to withstand the applied forces. Alternatively, or in addition, for portions of pipe 102 that remain above the surface of soil (e.g., when system 100 is used for process monitoring), the pipe wall can be considerably thinner. In certain embodiments, when system 100 is used for detection of species such as mold spores and/or indoor pollutants, pipe 102 may be absent entirely as discussed above. In some embodiments, the outside diameter of pipe 102 is typically approximately 35 mm. In certain embodiments, the outside diameter of pipe 102 is between 10 mm and 70 mm (e.g., between 15 mm and 60 mm, between 20 mm and 50 mm).

The inside diameter of pipe 102 can also be selected according to the sizes of the components housed within the pipe. In some embodiments, for example, the inside diameter of pipe 102 is typically approximately 22 mm. In certain embodiments, the inside diameter of pipe 102 is between 5 mm and 60 mm (e.g., between 10 mm and 50 mm, between 20 mm and 40 mm).

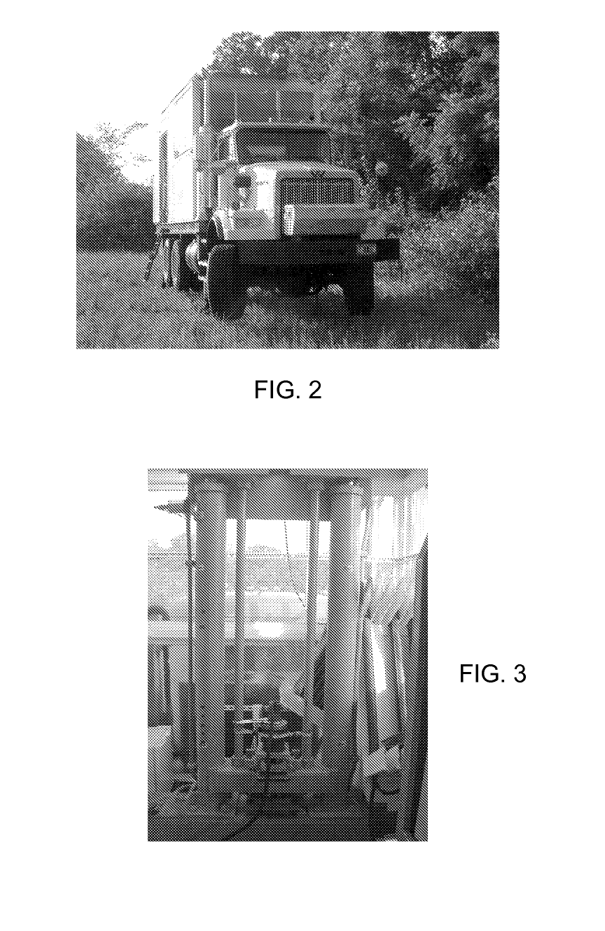

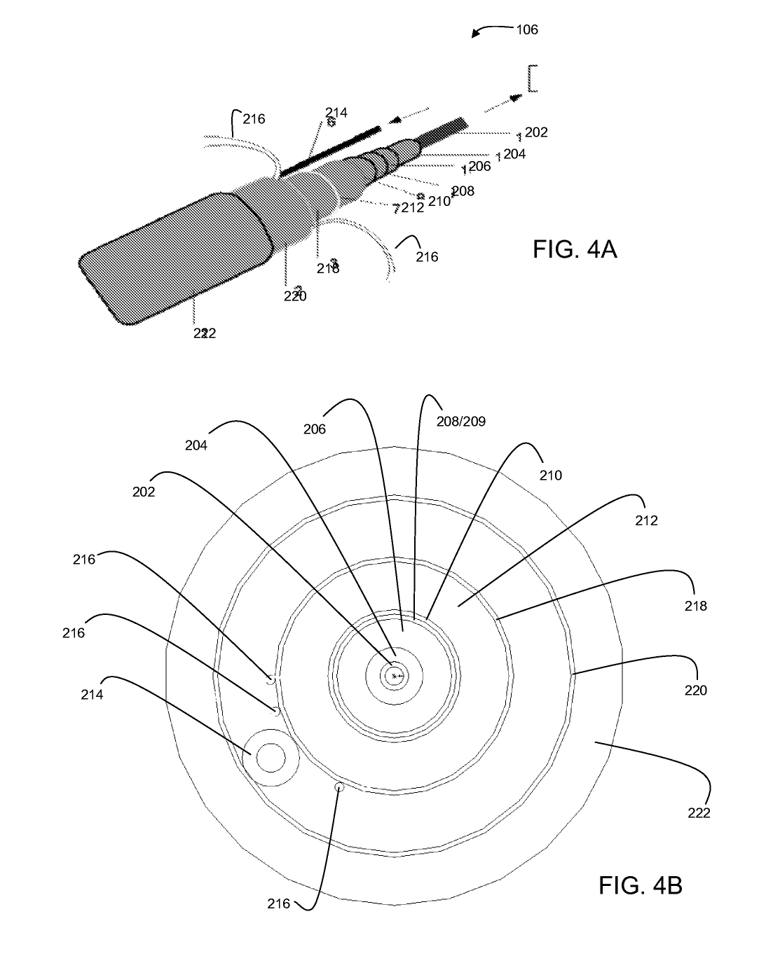

Pipe 102 can be inserted below ground level using a variety of techniques. In some embodiments, for example, pipe 102 can be inserted using a pushing apparatus (such as a pushing apparatus available from, for example, Geoprobe® Systems, Salina, Kans.). In certain embodiments, pipe 102 can be introduced below ground level using a cone penetrometer. FIG. 2 is a photograph showing a truck that houses a mobile cone penetrometer apparatus. FIG. 3 is a photograph showing the interior of the truck. Hydraulic lifts inside the truck raise the entire truck above ground level. The suspended weight of the truck then slowly drives pipe 102 into the ground as the hydraulic pressure is released.

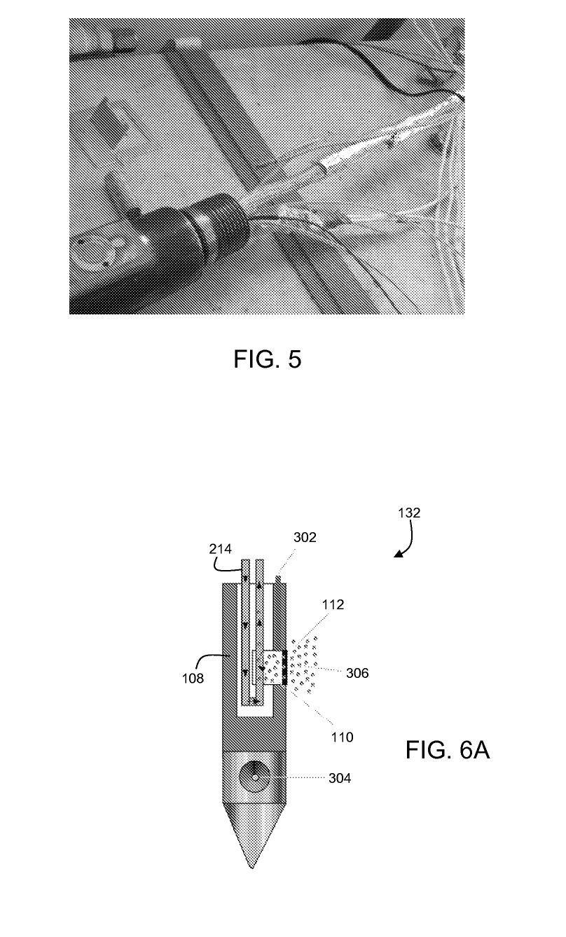

Transfer line 106 is constructed as a multi-layer, flexible tube that extends from inlet membrane 112 to analysis unit 116. FIG. 4A shows a schematic view of an embodiment of transfer line 106 with multiple layers peeled away, and FIG. 4B shows a schematic cross-sectional view of transfer line 106. The central compound-transporting conduit is a tube 202 formed of one or more metals such as stainless steel (e.g., a passivated stainless steel material such as Silcosteel®) sheathed in thermal insulation sleeves 204 and 206.

In general, a variety of different materials can be used to construct tube 202. In certain embodiments, as discussed above, tube 202 is formed of a stainless steel material such as Silcosteel®. In some embodiments, tube 202 is formed of a material such as nickel (e.g., electrical polished nickel). Tube 202 can also include coatings or linings formed on one or more surfaces of the tube, including coatings formed from materials such as glass and/or silicon (e.g., Siltek®, Sulfinert®. Thermal insulation sleeves 204 and 206 can also be formed from a variety of materials; exemplary materials include, for example, Silcosteel® and nickel, and such materials can include coatings formed of additional materials such as glass and/or Siltek®/Sulfinert®. At higher temperatures (e.g., for applications in which VOCs and SVOCs are thermally desorbed from a soil matrix), thermal insulation sleeves 204 and 206 can be formed from materials that can withstand elevated desorption temperatures; such materials include, for example, alumina-borica-silica (e.g., Nextel™) and other ceramic materials.

A layer of self-fusing silicone rubber tape 208 can be wrapped around the insulation sleeves, and a layer of high temperature fiberglass tape 210 can be made to surround the self-fusing silicone tape. Tape 210 primarily functions as thermal insulation; it is a woven material that is typically stable at temperatures of up to 600° C. Silicone rubber tape 208 functions as a water barrier, preventing moisture from entering the transfer line. Other materials can also be used to serve the same functions.

Two layers 212 and 218 of fiberglass glass cloth tape, typically stable to a temperature of about 300° C., can be applied to the high temperature fiberglass tape 210. In some embodiments, a layer of aluminum foil tape or another type of high temperature, reflective metal tape can also be applied between rubber tape 208 and fiberglass tape 210 (reflective tape 209 is a thin layer applied to the outer surface of rubber tape 208 in FIG. 4B) can also be applied. This layer of reflective tape reflects heat, ensuring that the transfer line remains stable and at an elevated temperature. In general, layer 212 of fiberglass cloth tape is implemented as a sleeve, while layer 218 is implemented as a wrapped coating. The number of layers of tape used depends largely on the environment in which the system is used. Moreover, layers of fiberglass tape can be replaced by other heat-resistant materials, such as ceramic-based tape.

Thermocouples 216 are positioned on the outer surface of cloth tape layer 218 and a Viton® carrier gas tube 214 is positioned such that it contacts the outer surface of cloth tape layer 218 and its central axis is aligned parallel to a central axis of tube 202. Although carrier gas tube 214 is formed of Viton® in FIGS. 4A and 4B, more generally, tube 214 can be formed from a variety of materials, including a variety of Teflon®-based materials, TFT, and/or flexible metal tubes formed of Silcosteel®.

A heat shrinkable polyolefin sleeve 220 secures the thermocouples and carrier gas tube 214 to the outer surface of tape layer 218, and a woven fabric layer 222 is applied to the outside of polyolefin sleeve 220. In certain embodiments, sleeve 220 can be formed of materials other than polyolefins, such as polytetrafluoroethylene and/or fiberglass.

In some embodiments, the length of transfer line 106 can be selected to test for the presence of organic contaminants positioned relatively deeply below the soil surface at a particular site. For example, the length of transfer line 106 can be 1 m or more (e.g., 2 m or more, 3 m or more, 5 m or more, 20 m or more, 40 m or more).

Transfer line 106 is configured to transport VOCs and SVOCs from sub-surface locations to analysis unit 116 for characterization. As described previously herein, during operation, processing unit 128 directs an electrical current to pass through transfer line 106 to control the temperature of transfer line 106 by resistively heating tube 202. Typically, for example, tube 202 is heated to a temperature of about 280° C. to prevent condensation of VOCs and SVOCs in transfer line 106. Further, as described above, processing unit 128 and control and monitor flow rates and pressures of gases transported by transfer line 106.

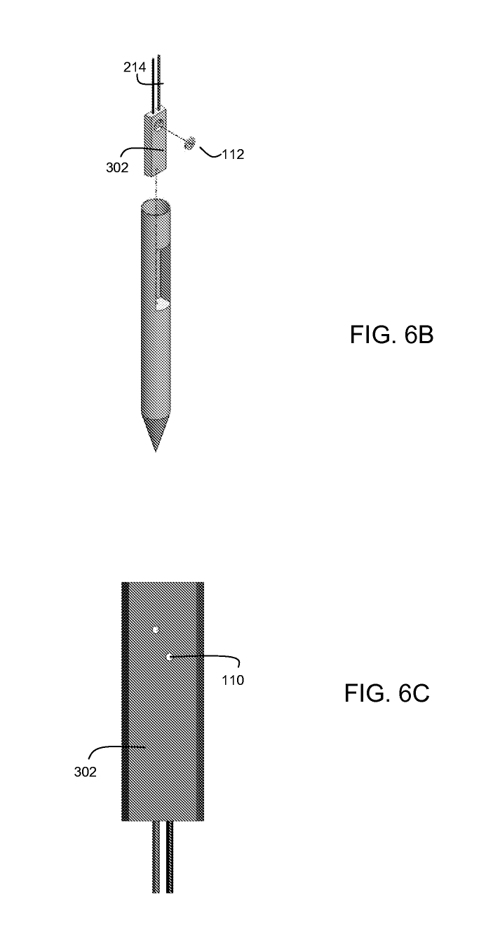

FIG. 5 shows a photograph of inlet probe 132. Sample collection port 110 forms an aperture in the body of second pipe 108, and inlet membrane 112 is positioned in the opening in collection port 110. Pipe 108 also includes one or more heater blocks (not shown in the photograph) for heating inlet probe 132, a connection port for connecting to transfer line 106, and a plurality of electrical conductors that connect to processing unit 128 for heating and monitoring the temperature of inlet probe 132. Typically, second pipe 108 has a length of about 33 cm, although pipes having different lengths can also be used.

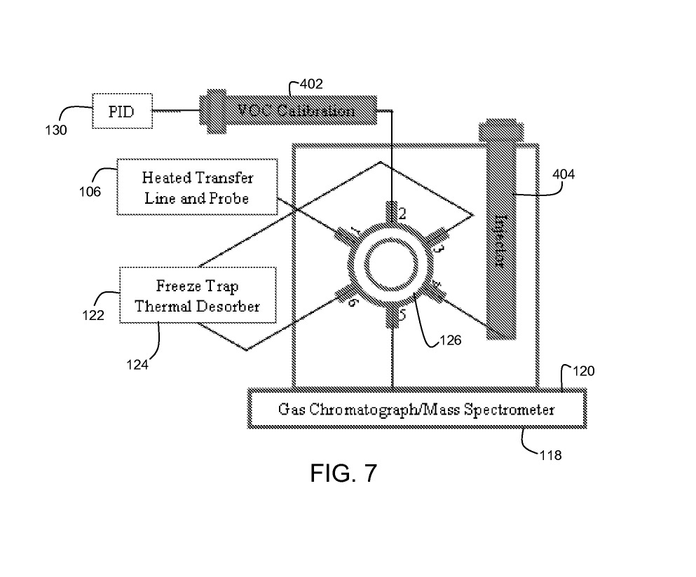

FIG. 6A shows a schematic view of inlet probe 132 and FIG. 6B shows an exploded view of the same probe. Collection port 110 is formed within pipe 208 of inlet probe 132. Inlet membrane 112 is positioned in the opening to collection port 110, and functions to selectively control the passage of different compounds through port 100. Carrier gas tube 214 extends through inlet probe 132 and supplies dry carrier gas for compound transport. Heater block 302 (shown in an expanded schematic view in FIG. 6C) is connected to processing unit 128 and heats inlet probe 132 during operation. Integrated within the tip of inlet probe 132 is a soil conductivity measurement tool 304 that is also connected to processing unit 128.

In the schematic view of FIG. 6C, inlet probe 132 does not include an inlet membrane 112. In general, the various components of system 100 can be used alone, or in conjunction with one another, depending upon the specific application, samples being analyzed, and/or site. Thus, for example, in some embodiments inlet probe 132 includes inlet membrane 112 and in some embodiments, inlet probe 132 does not include an inlet membrane. An inlet membrane may not be used, for example, when the concentration of water in the matrix material (e.g., soil) is relatively low, e.g., 5% or less. In addition, inlet probe 132 may not include a membrane for other applications such as detection of organic compounds in walls, ducts, and other enclosed spaces where the compounds may already be present in gaseous form. Alternatively, when system 100 is used in environments where the amount of water or steam present is relatively high (e.g., 10% or more by weight in a soil matrix), then an inlet membrane can be used to reduce or prevent damage to the lining of transfer line 106.

In certain embodiments, system 100 includes a freeze trap 122 and desorber 124 of the type described herein; typically, freeze trap 122 includes a tube or similar member in which VOCs and SVOCs are condensed, and desorber 124 is implemented as a coil that is wrapped around freeze trap 122. In this manner, both VOCs and SVOCs can be efficiently condensed and transported to GC unit 118 and MS unit 120.

In certain embodiments, system 100 includes a different type of freeze trap and/or desorber (or none at all). In general, freeze trap 122 and desorber 124 can be used to concentrate relatively dilute organic compounds in a stream of carrier gas from transfer line 106. Thus, for example, when the concentration one or more of the organic compounds of interest carried by transfer line 106 is so small that accurate detection and quantitative measurements are difficult, freeze trap 122 can be used to condense the organic compounds in liquid form inside the trap, concentrating them relative to their concentration in the carrier gas stream. Desorber 124 can be used to vaporize the condensed organic compounds to allow the more highly concentrated compounds to be detected and analyzed. Alternatively, when the concentration of organic compounds in the carrier gas stream from transfer line 106 is relatively high and/or very rapid detection of compounds is desired, system 100 may operate without a freeze trap or desorber.

In some embodiments, system 100 includes a multi-way valve 126 of the type described herein, and in some embodiments, system 100 includes a different type of valve. As an example, when inlet probe 132 does not include inlet membrane 112, the range of conditions in which system 100 can be used may be restricted to, for example, soils where the moisture content is 10% or less by mass, to avoid damage to transfer 106. As soils of interest may have moisture content that falls below this limit, system 100 can still be used in a wide variety of situations.

Soil conductivity measurement tool 304 can be used to measure the water content of soil into which inlet probe 132 is inserted. Water content information can be important for a number of reasons. For example, adsorbed VOCs and SVOCs in soil will mitigate at different rates based on the water content in the soil. Accordingly, water content information can be used to control the temperature of inlet probe 132, and to provide messages and/or warnings to a system operator. Further, soil moisture content influences the types of remediation reagents used and their effectiveness. Thus, measurement of water content can provide important information in advance of a remediation program.

In certain configurations, transfer line 106 includes an internal coating that is sensitive to, and degraded by, water. To maintain proper functioning of transfer line 106, it is therefore important to ensure that the amount of water introduced into transfer 106 is minimized. As discussed further below, one method for accomplishing this is to use a specially constructed inline membrane. More generally however—whether the system is used with or without a special membrane—soil conductivity measurement tool 304 provides information about the environment into which inlet probe 132 is introduced. This information can be used by a system operator to judge whether transfer line 106 is at risk due to the presence of excess moisture in the soil and whether, for example, installation of a special water-impermeable membrane is appropriate. Typically, the coating in transfer line 106 is degraded sufficiently to destroy the transfer line when the moisture content of the soil reaches about 15%, for example. Thus, measurement of water content can be used to provide messages and/or warnings to a system operator regarding the nature of the environment in which inlet probe 132 operates and whether transfer line 106 is at risk; such information can be particularly important when inlet probe 132 is used without a water-impermeable membrane.

During operation, inlet probe 132 is typically heated to about 400° C. by heater block 302 to thermally desorb VOCs and SVOCs 306 that are bound to naturally occurring matrix materials in the soil. In this disclosure, the term “matrix materials” used in reference to soil refers to materials—typically solids, but also liquids—that are not analyzed by system 100. Matrix materials can be organic and/or inorganic, and can function as support surfaces onto which VOCs and SVOCs of interest are adsorbed. Typically, matrix materials are relatively porous and have relatively large surface area for adsorption of VOCs and SVOCs.

For soils with water concentrations of less than 10%, the soil in contact with inlet probe 132 will reach a temperature of about 300° C. after 5 minutes of heating. VOCs and SVOCs desorb from soil matrix materials under these conditions. Dry carrier gas (e.g., nitrogen) circulating through gas tube 214 is injected into the soil at a selected flow rate to collect the desorbed VOCs and SVOCs. As an example, dry carrier gas can be injected into the soil at a flow rate of about 5.5 mL/min. More generally, carrier gas flow rates can be between 2.0 mL/min. and 10.0 mL/min. (e.g., between 3.0 mL/min. and 9.0 mL/min, between 4.0 mL/min. and 8.0 mL/min.).

A vacuum pump housed within analysis unit 116 provides a reduced-pressure environment within the interior region of pipe 108, such that desorbed VOCs and SVOCs 306 enter pipe 108 through inlet membrane 112. Once inside pipe 108, VOCs and SVOCs 306 (in the vapor phase) are transported to analysis unit 116 through transfer line 106, which is coupled to pipe 108. Once inside analysis unit 116, VOCs and SVOCs 306 are analyzed in GC and MS units 118 and 120, respectively, or directed to PID 130.

Heating of the various components of system 100, directing recovered VOCs and SVOCs to different instrument units, and analysis of measurement results from the different instruments units is performed by processing unit 128. Processing unit 128 can be configured to accept commands from a system operator to control various system parameters such as carrier gas flow rates, heating rates, and sample collection times. In some embodiments, processing unit 128 can be configured for fully automatic operation during which no intervention on the part of a system operator is typically required.

The GC unit 118 and the MS unit 120 permit accurate characterization of different VOCs and SVOCs recovered from subsurface locations within the soil. However, analysis of VOCs and SVOCs using these instruments only proceeds after the VOCs and SVOCs have been first detected and collected. To detect VOCs and SVOCs, PID 130 is used to monitor photoionization signals from compounds that are transported into analysis unit 116 as system 100 is advanced deeper into the soil. For example, during advancement of system 100, compounds that enter pipe 108 through inlet membrane 112 are transported by transfer line 106 to analysis unit 116. Multi-way valve 126 is configured (e.g., by processing unit 128) to direct the compounds to PID 130. In this manner, PID 130 continuously records photoionization signal as system 100 (and more specifically, inlet probe 132) is advanced into the soil.

If no VOCs or SVOCs are present in the soil, the “compounds” that are transported to analysis unit 116 consist principally of nitrogen gas. Typically, background signals are determined from measured photoionization signals from the first 5-10 cm of soil (where no VOCs and/or SVOCs are expected to be present, so that the measured photoionization signal is due largely to the nitrogen carrier gas), and are subtracted from subsequent photoionization signals corresponding to compounds recovered from deeper locations in the soil. Negative PID signals indicate no detectable organic compounds (e.g., VOCs and/or SVOCs). Positive PID signals at a particular inlet probe depth correspond to detectable quantities of VOCs and SVOCs at that depth. By measuring PID signals as a function of depth, a depth distribution profile of organic contaminants (e.g., coal tar contaminants) for a particular plot of soil can be constructed in which contaminant concentrations (which are related to the magnitude of the measured PID signals) can be correlated with depth below the surface, and with lateral position at the surface (e.g., two-dimensional position in the plane of the surface).

When organic contaminants are detected at a particular depth, the contaminants can be sampled and analyzed to determine the chemical identities of the contaminants via GC-MS methods. This analysis yields a conceptual site model in which concentrations of specific contaminants are expressed as a function of three-dimensional geographic locations below the surface of the soil. For coal tar- and petroleum-contaminated sites, where excavation of contaminated soil is often the preferred remediation remedy, estimates of the volume of contaminated soil and the nature of the contaminants are valuable for estimating cleanup costs.

The methods disclosed herein can also be used to identify particular “hot spots” (e.g., large concentrations of sub-surface organic contaminants), locate the boundaries of such hot spots, and to track migration of contaminants underneath the soil surface. For example, by repeating measurements at a selected site over a period of months or years, migration of sub-surface organic contaminants can be mapped. Because different contaminants migrate at different rates, GC-MS analysis can be used together with measured PID signals to map migration patterns and rates for specific organic contaminants, information that is both valuable for assessing the ongoing contamination risk to a particular site, and difficult to obtain using other means.

Additional aspects and features of system 100 are disclosed, for example, in U.S. Pat. No. 6,487,920, the entire contents of which are incorporated herein by reference.

### Inlet Membranes

Inlet membrane 112 performs a number of important functions in system 100. Inlet membrane 112 prevents a substantial quantity of soil matrix material from entering pipe 108 and transfer line 106. This matrix material is not amenable to analysis by GC-MS methods and would contaminate system 100. Instead, system 100 is configured so that during operation, VOCs and SVOCs that are bound to such matrix material are thermally desorbed from the matrix material at the location of membrane 112 (e.g., adjacent to inlet probe 132), and only the VOCs and SVOCs (and not the matrix material) are transported to analysis unit 116. Thus, inlet membrane 112 also functions to allow VOCs and SVOCs to pass through port 110 and into the interior of pipe 108. This important filtering function ensures that detectable quantities of these compounds can be delivered to analysis unit 116.

Inlet membrane 112 is also configured to prevent water from entering pipe 108 and transfer line 106. Transfer line 106, in particular, has an internal coating that is degraded by water vapor. If large quantities of water vapor were permitted to enter transfer line 106 the usable lifetime of the tube would be significantly reduced, because it is the internal coating material that is resistively heated to maintain the elevated temperature in transfer line 106. Accordingly, inlet membrane 112 is designed to prevent the passage of water vapor to retard degradation of transfer line 106.

To prevent the passage of water, inlet membrane 112 is typically formed of a fluorinated polymer-coated support material; the high hydrophobicity of the fluorinated polymer discourages passage of water through the support material. In general, a variety of different types of fluorinated polymers can be used in inlet membrane 112. As an example, Teflon®-coated support materials can be used in inlet membrane 112.

Standard Teflon®-coated support materials, however, can only be used in limited circumstances in connection with system 100. In particular, the water permeability of standard Teflon®-coated support materials is, in general, not low enough, particularly at elevated temperatures (e.g., 250° C. or more) to prevent significant quantities of water from entering transfer line 106 when the surrounding soil has high concentrations of water. Thus, for example, it has been discovered that the application of standard Teflon®-coated support materials is limited to investigation of soils having water concentrations of about 10% or less. For soils with water concentrations in excess of this amount, unacceptable quantities of water are observed to pass into transfer line 106.

Moreover, the thermal stability of Teflon®-coated support materials depends in large measure on the nature of the Teflon® coating applied to the support material. Standard Teflon® coated support materials are capable of being heated to approximately 120° C. before they break down. At a temperature of 120° C., certain VOCs can be thermally desorbed from soil matrix materials and collected by inlet probe 132. However, other VOCs and most SVOCs do not undergo efficient thermal desorption at these relatively low temperatures. As a result, standard Teflon®-coated matrices are typically limited to applications involving collection and characterization of VOCs.

Temperatures of approximately 300° C. are generally used to ensure that as many of the VOCs and SVOCs of interest as possible are desorbed, collected, and transferred to analysis unit 116 in the vapor phase. As discussed above, heating the soil surrounding inlet probe 132 to a temperature of 300° C. typically involves heating inlet probe 132 to a temperature of approximately 400° C. while subsurface measurements are performed. Standard Teflon®-coated matrices generally do not survive such conditions.

To address the above difficulties, a specialized inlet membrane 112 was constructed. Inlet membrane 112 was prepared from a stainless steel mesh support material with a pore size of between 20 microns and 200 microns. Suitable stainless steel mesh support materials can be obtained, for example, from Belleville Wire Cloth Company, Cedar Grove, N.J.

The mesh support material was scoured with a wire brush to remove particulates, and then sonicated in toluene for about 30 minutes. Then, using a Pasteur pipette, drops of PTFE TE3859 dispersion (obtained from Fuel Cell Earth, Stoneham, Mass.) were applied to one side of the mesh and allowed to disperse until the mesh was completely covered. A small paintbrush was used to assist in spreading the dispersion evenly. The mesh was allowed to air-dry for 15 minutes, and then was placed in an oven and cured under Ar gas. Curing was achieved by baking at a temperature of 100° C. for 30 minutes, followed by baking at 200° C. for 30 minutes, and then finally baking at 300° C. for 60 minutes.

After the first side of the mesh fully cured, the other side of the mesh was spray coated with PTFE TE3859 dispersion using a low pressure spray gun. The spray-coated side was dried for 15 minutes, and then cured using the same temperature baking process described above. The process of spray coating, drying, and baking was then repeated two additional times on the second side of the support material. After completion of the coating procedure for the second side, the second side was scraped with a razor to remove excess Teflon® coating material. Finally, the entire coated mesh was cured for additional 24 hours by baking at a temperature of 300° C.

The resulting inlet membrane 112 was capable of being heated to significantly higher temperatures than conventional Teflon®-coated support materials without undergoing significant degradation. In particular, inlet membrane 112 prepared in the manner disclosed above can be heated to a temperature of 140° C. or more (e.g., 160° C. or more, 180° C. or more, 200° C. or more, 240° C. or more, 280° C. or more, 300° C. or more, 350° C. or more, 400° C. or more) without undergoing significant degradation. Degradation by-products can be measured for example, by installing inlet membrane 112 in inlet probe 132, and then heating inlet probe 132 (including inlet membrane 112) to a temperature of 300° C. for a period of between 8 and 10 hours per day over a period of three months while carrier gas is swept through transfer line 106. During this period of heating, any degradation by-products from membrane 112 are carried via transfer line 106 to analysis unit 116, where they are detected by GC and MS units 118 and 120, respectively. Tests conducted as above on inlet membranes prepared using the methods disclosed herein yielded no detection of degradation by-products from the membranes during the three month testing period. This testing period was considered to be a suitable test of long-term membrane resistance to failure, because during field use, pipe 102 and its associated components (e.g., membrane 112) are frequently damaged when they are forced through the soil and they strike hard objects such as subsurface rocks. As a result, a membrane exhibiting resistance to temperature-induced breakdown over a testing period of three months is therefore unlikely to exhibit any breakdown throughout the duration of its useful lifetime when making field measurements.

Inlet membrane 112 can be used to filter VOCs and SVOCs from soils that contain water, because the pores in membrane 112 preclude water molecules from passing through the membrane. Thus, in general terms, inlet membrane 112 functions as a filter to prevent the passage of water from one region (e.g., in the soil) to a second region (e.g., transfer line 106), but should also be robust enough to withstand the rigors of the harsh testing conditions in terms of very high temperatures, abrasion, and pressure.

More particularly, as explained above, when VOCs and SVOCs are collected from the soil at temperatures of 300° C. or more, the coating that lines transfer line 106 is susceptible to degradation by water molecules. In practical terms, without the inlet membrane, transfer line 106 can only be used in environments where water concentrations are sufficiently low such that the lining of transfer line 106 is not rapidly destroyed. For collection of VOCs and SVOCs from soil, this establishes an upper limit of approximately 10% on the amount of water that can be present in the soil without leading to rapid degradation of transfer line 106′s coating.

Thus, inlet membrane 112 significantly expands the range of soils in which system 100 can be used to perform measurements by acting as a filter to prevent water molecules in the soil from passing into transfer line 106. By preventing this passage of water molecules, the operational lifetime of transfer line 106 is extended, and system 100 can be used in a significantly greater variety of conditions. For example, inlet membrane 112 can be used to filter organic compounds such as VOCs and SVOCs from soils with water concentrations of 15% or more (e.g., 20% or more, 25% or more, 35% or more, 50% or more, or even more), including sludgy soils, trapped pools of water, and more generally, from any body of water. In some embodiments, system 100 equipped with an inlet membrane 112 prepared as disclosed above can be used in pools that are composed nearly entirely of water, thus allowing system 100 to move through soil without risking damage due to trapped pools of water that may be encountered. In this way, inlet membrane 112 permits system 100 to be used in a variety of circumstances that would not be possible with an inlet membrane formed of conventional materials.

The effectiveness of inlet membrane 112 at preventing the passage of water molecules results from a combination of the nature of the mesh support material used, the thickness of the Teflon®-based coating applied to the mesh, and the chemical nature of the Teflon®-based coating. These factors combine to yield a membrane having a particular stability as a function of temperature (e.g., with as little as no measureable degradation at temperatures of at least 300° C.). These factors also combine to yield a membrane having an average pore size that is small enough to prevent the passage of water through the membrane, and large enough to permit the passage of VOCs and SVOCs.

A variety of different stainless steel mesh materials can be used as the mesh support material. In some embodiments, the mesh support material is relatively stiff and resists deformation. Support materials of this type are particularly well-suited for producing inlet membranes, which are typically inserted forcefully into the ground and therefore are subject to significant mechanical stress. In certain embodiments, the mesh support material can be relatively more flexible; such support materials are suitable when the membranes produced from them are used in applications where they are intentionally deformed.

The weave pattern and opening size of the mesh support material can be selected to achieve a particular average pore size in membrane 112. For example, in some embodiments, the opening size (e.g., the average maximum dimension of the openings in the material) can be 20 microns or more (e.g., 40 microns or more, 80 microns or more) and/or 200 microns or less (e.g., 150 microns or less, 100 microns or less). As examples, the weave pattern of the mesh can include a Dutch weave, a Twill weave, a plain weave, and/or other types of weaves as well.

The type of Teflon®-based material that is used to coat the mesh support material should be selected to create a membrane that is both stable at temperatures of 300° C. or more, and includes pores small enough to prevent the passage of significant quantities of water molecules. While PTFE TE3859 has been used in the procedures disclosed herein to prepare inlet membrane 112, other fluorinated polymer-based coatings, including coatings based on certain other Teflon® materials, can also be used to prepare membranes with suitably high temperature stability and low water permeability. To achieve suitable pore sizes in membrane 112, the thickness of the applied Teflon®-based coating can be between 5 microns and 60 microns (e.g., between 10 microns and 50 microns, between 20 microns and 40 microns).

As discussed above, membranes prepared according to the methods disclosed herein were tested to ensure that no degradation occurred upon heating over extended periods of use. The prepared membranes were also subjected to a filtration test to determine whether they acted as suitable filters to exclude water. In the first step of the filtration test, a candidate membrane is heated to a temperature of 300° C. and water is poured over the membrane. The membrane is observed to ensure that no water passes through the membrane.

If water is not observed to pass through, the second step of the filtration test is performed in which a source of steam is positioned in proximity to the candidate membrane still heated to 300° C., and the membrane is observed to determine whether any of the steam passes through.

If steam is not observed to pass through, the third step of the filtration test is performed in which the candidate membrane, still at a temperature of 300° C., is exposed on one side of the membrane to VOCs. A detector positioned on the other side of the membrane is configured to detect organic molecules passing through the membrane. If the VOCs are observed to pass readily through the membrane, the membrane is deemed to be suitable for use in system 100.

The description of membrane 112 above has largely focused on the use of the membrane as a component of inlet probe 132. More generally, however, the methods disclosed herein can be used to prepare Teflon®-based membranes in a variety of different shapes and for a variety of applications. For example, the methods can be used to prepare sheets of membranes that can subsequently be processed mechanically (e.g., by cutting or dicing) to yield membranes of any desired shape.

In addition to measurement of VOCs and SVOCs as described herein, such membranes can also be used for other applications. As an example, the membranes disclosed herein can be used for monitoring process flows and/or indoor air for contamination in steam-filled and/or humid environments. The ability of the membranes to prevent passage of water while at the same time permitting passage of organic compounds permits efficient detection of such compounds without the confounding and contaminating effects of water. As a another example, the membranes disclosed herein can also be used for outdoor pollution monitoring, as they permit organic compounds such as VOCs and SVOCs to be detected in the presence of relatively large concentrations of air- or soil-borne water. Other exemplary applications include process monitoring in manufacturing (e.g., process stream monitoring), mechanical and/or aeronautical exhaust monitoring, and forensic detection (e.g., police and fire investigations).

### Multi-Way Inlet Valves

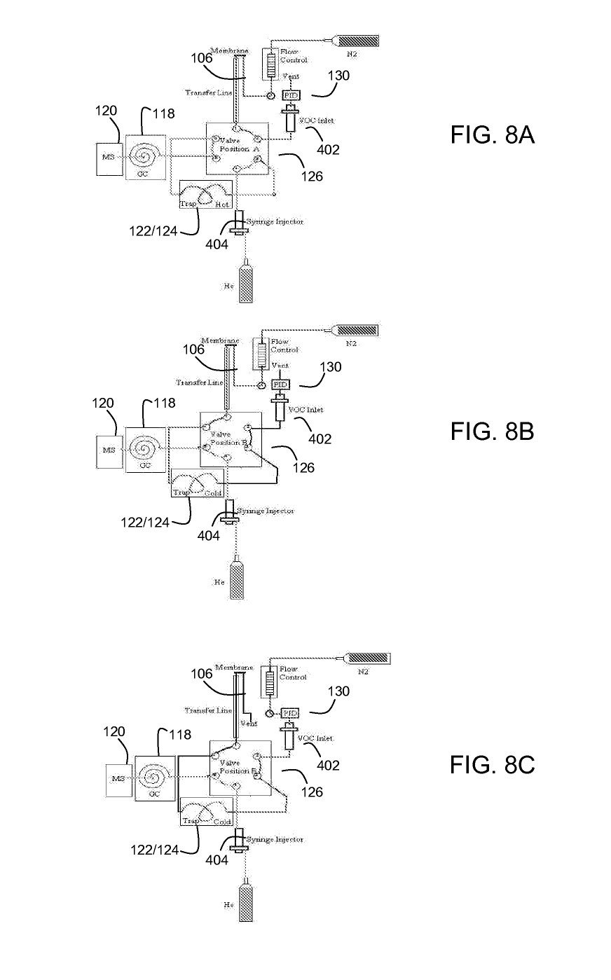

As disclosed above, multi-way inlet valve 126 is configured to direct recovered VOCs and SVOCs to the GC and MS units 118 and 120 for analysis, or to PID 130 for detection and measurement of contaminant concentrations. Multi-way inlet valve 126 also provides an inlet port for introducing carrier gas (e.g., nitrogen) into system 100. FIG. 7 shows a schematic diagram of multi-way inlet valve 126. Inlet valve 126 includes six ports labeled “1” through “6” in FIG. 7. Port 1 is connected to transfer line 106 and inlet probe 132. Port 2 is connected to PID 130 and to VOC calibration unit 402 in series. Valve 126 is connected to freeze trap 122 and thermal desorber unit 124 through Ports 3 and 6. Port 4 is connected to syringe injection unit 404 that extends outside analysis unit 116, and permits connection of an external gas source (e.g., a source of a gas such as helium, neon, argon, and/or nitrogen) to system 100. Port 5 of valve 126 is connected to GC unit 118 and to MS unit 120.

Gas flow within the system can occur in multiple directions. As will be discussed in greater detail below in connection with ports 1-6, in some embodiments, gas flows from PID 130 through VOC calibration unit 402, through freeze trap 122, and into transfer line 106. Alternatively, in certain embodiments, gas flows in the opposite direction from transfer line 106 through freeze trap 122, through VOC calibration unit 402, and into PID 130.

The six ports in valve 126 are connected in pairs to permit different detection, analysis, and calibration steps to be performed by system 100. Flow paths are adjusted under the control of processing unit 128 during different measurement stages as a contaminated site is explored using the system.

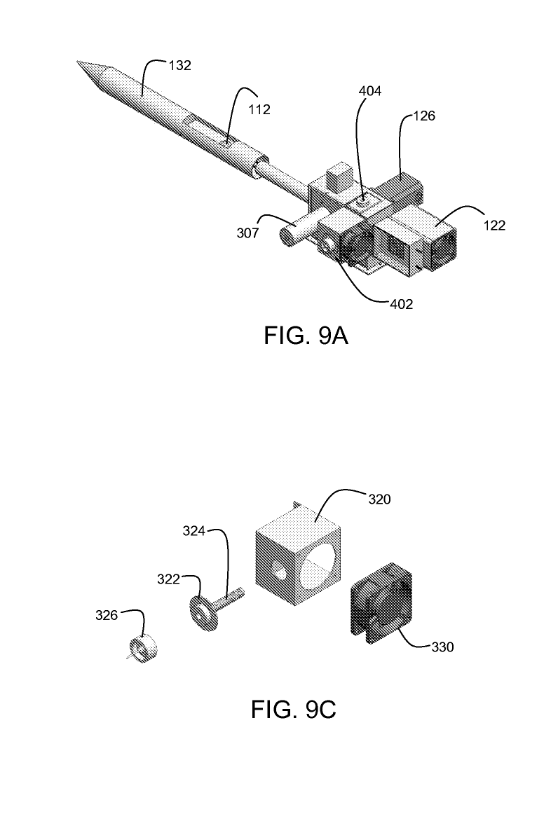

FIGS. 8A-8C are schematic diagrams that show different configurations of multi-way inlet valve 126 at different stages of contaminant characterization and measurement. As disclosed above, when inlet probe 132 is advanced through soil during exploratory activity, transfer line 106 is in fluid connection with PID 130. PID 130 measures photoionization signal from the compounds that are transported to analysis unit 116 from inlet probe 132 through transfer line 106. A positive photoionization signal indicates the presence of organic contaminants in the soil. To achieve a fluid connection between transfer line 106 and PID 130, multi-way valve 126 is adjusted by processing unit 128 to the connection configuration shown in FIG. 8A. In this configuration, ports 1 and 2 are connected in valve 126 so that compounds traveling through transfer line 106 and into multi-way valve 126 through port 1 are directed into PID 130 through port 2. Ports 3 and 4 are also directly coupled so that helium gas introduced through syringe injection unit 404 coupled to port 4 is directed into freeze trap 122 connected to port 3. Ports 5 and 6 are also directly coupled so that the helium gas that flows through freeze trap 122 re-enters valve 126 through port 6, and then flows into GC unit 118 and MS unit 120 through port 5.

PID 130 remains in fluid connection with transfer line 106 until a spike in the photoionization signal (e.g., a positive photoionization signal) is measured, indicating the presence of organic contaminants in the soil under investigation. When a positive signal is measured, processing unit 128 adjusts multi-way valve 126 to the configuration shown in FIG. 8B. In FIG. 8B, ports 1 and 6 are connected so that compounds (e.g., VOCs and SVOCs) transported by transfer line 106 enter valve 126 through port 1, and then flow directly into freeze trap 122 through port 6. Once inside freeze trap 122, the compounds are condensed and trapped inside a chemically inert coiled tube. During trapping of the compounds, ports 4 and 5 in valve 126 are connected so that helium gas introduced through syringe injection unit 404 flows through GC unit 118 and MS unit 120.

After the compounds have been trapped in freeze trap 120, processing unit 128 again switches the port connection configuration in valve 126 back to the configuration shown in FIG. 8A. In this configuration, helium gas enters valve 126 through port 4 and is coupled into freeze trap 122 through valve port 3. The helium gas flows through freeze trap 122 and exits through port 6, re-entering valve 126. The helium gas enters GC unit 118 through port 5 of valve 126, which is directly coupled to port 6. The flowing helium gas functions as the carrier gas for GC-MS analysis of the trapped VOCs and SVOCs. As the helium gas flows through freeze trap 122, the freeze trap is resistively heated under the control of processing unit 128, vaporizing the trapped organic compounds. The vaporized organic compounds are swept through GC unit 118 and MS unit 120 by the flowing helium gas, where they are analyzed.

Multi-way valve 126 also permits on-line calibration of GC unit 118 and MS unit 120 for both VOCs and SVOCs. During calibration, processing unit 128 adjusts valve 126 to the configuration shown in FIG. 8C. In this configuration, ports 4 and 5 are coupled so that reference standards can be introduced directly into GC unit 118 and MS unit 120 through syringe injection unit 404. SVOC calibration is performed by syringe injection of one or more standards into injection unit 404; the standards are analyzed by GC unit 118 and MS unit 120, and the operating parameters of these units are adjusted according to the expected results.

To perform VOC calibration, one or more VOC reference compounds are purged from an aqueous medium and trapped onto an adsorbent-packed tube. The tube is placed in VOC calibration unit 402 (or is connected to VOC calibration unit 402, e.g., by removing PID 130 and connecting the tube to VOC calibration unit 402), and with multi-way valve 126 in the configuration shown in FIG. 8B, the VOC reference standard(s) enter freeze trap 122 through port 3, where they are trapped. Valve 126 is then switched to the configuration shown in FIG. 8A by processing unit 128, and thermal desorber 124 is activated by processing unit 128 to desorb the reference standard(s) from the freeze trap. At the same time, helium entering valve 126 through port 4 flows through freeze trap 122 from port 3 through to port 6, sweeping along the one or more desorbed VOC reference standards. The helium gas and VOC standard(s) enter GC unit 118 and MS unit 120 through port 5 of valve 126. The standard(s) is/are analyzed, and the operating parameters of units 118 and 120 are adjusted according to the results of the analysis.

The configuration of components shown in FIGS. 7 and 8A-8C is highly flexible, and permits calibration of system 100 using a variety of techniques. As described above, one such technique permits introduction of one or more reference standards packed onto an adsorbed tube by connecting the tube to VOC calibration unit 402. In certain embodiments, no adsorbent tube is used for calibration. For example, reference standards can be injected directly into VOC calibration unit 402, where they are eventually conveyed to and trapped by freeze trap 122. By switching valve 126 from the configuration shown in FIG. 8B to the configuration shown in FIG. 8A, the trapped reference standard(s) can then be conveyed to and analyzed by GC unit 118 and MS unit 120, as described above.

Alternatively, in some embodiments, one or more reference standards can be introduced through syringe injection unit 404. With valve 126 in the configuration shown in FIG. 8A, the injected standards are first condensed and trapped by freeze trap 122, and then later desorbed for analysis by GC unit 118 and MS unit 120.

The use of multi-way valve 126 therefore enables a variety of different methods for calibrating system 100 using reference standards. Both VOC and SVOC reference standards can be introduced into the system via injection (e.g., through units 402 and/or 404) and/or using an adsorbed tube (e.g., coupled to unit 402). Further, multi-way valve 126 permits calibration using reference standards in a variety of different physical states, including solids, liquids, and gases. This flexibility is important, particularly in situations when work at a site necessitates calibration of system 100 using available standards, which may be present in a variety of different states. Moreover, the best state for establishing an accurate calibration for a particular analyte may differ relative to other possible analytes. The versatility of multi-way valve 126 in combination with the different methods of introducing compounds disclosed above allows any collection of compounds in the same or different states (e.g., a soil matrix) to be introduced into system 100 simultaneously. To facilitate analysis of such compounds, multi-way valve 126 allows each of the detectors in system 100 to be configured serially (e.g., in-line with one another) to minimize the amount of either a standard or recovered compound necessary for achieving accurate analysis results.

In some embodiments, a 10-port multi-way valve can be used in the systems disclosed herein to achieve similar functionality. In the 10-port valve, port 1 is connected to an injector, ports 2 and 5 are connected to freeze trap 122 and thermal desorber 124 (freeze trap 122 collects compounds flowing from the VOC calibration unit 402), port 3 is vented to atmosphere, port 4 is connected to VOC calibration unit 402, port 6 is connected to GC and MS units 118 and 120, ports 7 and 10 are connected to a second freeze trap (with the second freeze trap collecting the compounds flowing from transfer line 106), port 8 is connected to transfer line 106, and port 9 is connected to PID 130. In a first valve position, materials desorbed from soil that pass through inlet membrane 112 and into transfer line 106 flow into PID 130. At the same time, gas flow also occurs through VOC calibration unit 402 and into freeze trap 122, and from the injector through the second freeze trap to GC and MS units 118 and 120. Accordingly, when the 10-port multi-way valve is in the first position, the system permits syringe injection, freeze trapping of VOCs from VOC calibration unit 402, thermal desorption from the second freeze trap, and monitoring by PID 130 of analytes from transfer line 106.

In a second valve position, three additional gas flow paths are defined. First, gas (and desorbed analytes) flows from transfer line 106 to the second freeze trap. Second, gas flows from VOC calibration unit 402 to atmospheric vent. Third, gas flows from the injector coupled to GC unit 118 through freeze trap 122 and into GC and MS units 118 and 120. When the 10-port multi-way valve is in the second position, the system permits freezing of analytes from the transfer line onto the second freeze trap and desorption of VOC reference standards from freeze trap 122. In general, syringe injection of compounds (e.g., SVOC reference standards) is performed with the 10-port multi-way valve in the first position, and monitoring of analytes from transfer line 106 using PID 130 is also performed with the valve in the first position. Calibration of the system with VOC reference standards is performed by first introducing the standards with the valve in the first position, and then completing the analysis via GC and MS units 118 and 120 with the valve in the second position. In contrast, analysis of analytes from freeze trap 106 begins with the valve in the second position (so that the analytes are trapped in the second freeze trap), and is completed using GC and MS units 118 and 120 with the valve in the first position.

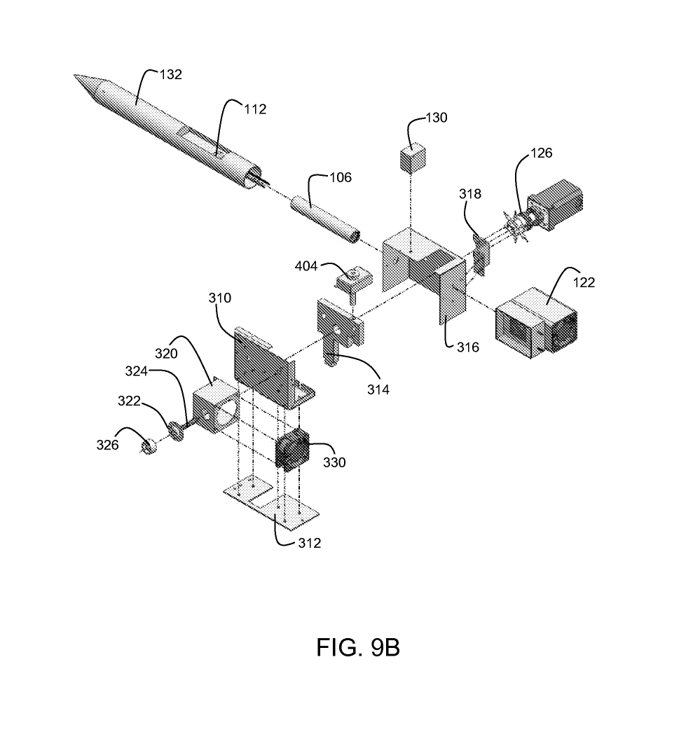

FIG. 9A is a schematic view of an integrated assembly that includes inlet probe 132 (with inlet membrane 112), multi-way valve 126, and low temperature freeze trap 122 (which can, in some embodiments, also include an integrated or connected thermal desorber 124). FIG. 9B shows an exploded view of a portion of the assembly of FIG. 9A. Within the assembly, base 310 is attached to base mount 312, and encloses heating block 314 which heats syringe injector 404 and multi-way valve 126. Mounted atop base cover 316 is PID 130. Also mounted to cover 316 is multi-way valve 126 via valve mount 318, and freeze trap 122. The assembly also includes CO₂ freeze trap 307 and VOC calibration unit 402. CO₂ freeze trap 307 can be used in combination with, or as an alternative to, freeze trap 122. Thus, for example, in the preceding discussions of the connections between components through multi-way valve 126, freeze trap 307 can be connected in place of, or in addition to, freeze trap 122. VOC calibration unit 402, which is shown in more detail in the exploded view of FIG. 9C, includes an injector mount 320, fan 330, wrapped cylindrical condenser 324, injector screw 322, and injector screw top 326.

Although VOC calibration unit 402 and multi-way valve 126 define flow paths in system 100 that permit reference standards to be analyzed and analytes from transfer line 106 to be detected by PID 130, applications of VOC calibration unit 402 and multi-way valve 126 are not limited to use merely in connection with system 100. VOC calibration unit 402 and multi-way valve 126 can be used with a variety of instruments for performing flow-based calibration and analysis of compounds, and provide the same advantages in other systems as have been described above in connection with system 100. Alternative applications in which VOC calibration unit 402 and/or multi-way valve 126 can be used include indoor and/or outdoor air monitoring, process monitoring in manufacturing (e.g., process stream monitoring), mechanical and/or aeronautical exhaust monitoring, and forensic detection (e.g., police and fire investigations).

### Low Temperature Freeze Traps

Field use of system 100 presents a number of unique challenges owing to the variable and relatively harsh environmental conditions that are typically encountered outside the laboratory. One such challenge is maintaining efficient operation of freeze trap 122. As disclosed above, freeze trap 122 condenses and traps both VOCs and SVOCs that are transported to analysis unit 116 by transfer line 106. The trapped VOCs and SVOCs are then re-vaporized and carried together to the GC and MS units 118 and 120 for analysis. By trapping both VOCs and SVOCs at the same time, freeze trap 122 permits both classes of compounds to be analyzed together, resulting in shorter analysis times (because VOCs and SVOCs are analyzed simultaneously rather than in series). As described herein, system 100 also typically includes thermal desorber 124 integrated with, or connected to, freeze trap 122. Freeze trap 122 and thermal desorber 124 allow VOCs and SVOCs to be collected via condensation and concentrated relative to their concentrations in the carrier gas during transport through transfer line 106. Then, once the organic compounds have been concentrated, they can be desorbed and efficiently conveyed directly to GC and MS units 118 and 120 for analysis. This streamlined and efficient approach is particularly useful when coupled with in situ sample collection (e.g., subsurface sampling), and permits dramatically increased sample analysis throughput rates and reduction of waiting times by field personnel for analytical data. Moreover, trapping of the VOCs and SVOCs permits concentration of trace level compounds prior to injection into the GC and MS units, improving the detection sensitivity of the analysis for each class of compound.

The systems and methods disclosed herein for simultaneous concentration and analysis of VOCs and SVOCs are in contrast to conventional methods of analysis of VOCs and SVOCs. Conventionally, VOCs are analyzed by recovering organics from water (even though soils may have been collected for analysis) and SVOCs are analyzed after extraction with an organic solvent. In contrast, the systems and methods disclosed herein permit both VOCs and SVOCs to be collected from soil and water and then analyzed using the same system at substantially the same time.

The design of a suitable freeze trap is made more difficult because freeze trap 122 is typically mounted to the top surface of GC unit 118 due to space constraints within system 100 (which is configured for mobile use in the field). GC unit 118 includes an internal oven that maintains an elevated temperature in GC unit 118 to ensure that compounds undergoing analysis do not condense prematurely on the GC column. The GC oven generates a considerable amount of heat, which influences the lowest temperature that freeze trap 122 can achieve. It has been discovered, for example, that a single-stage Peltier freeze trap can maintain a temperature of only −8° C. during operation when positioned atop GC unit 118.

To more efficiently trap both VOCs and SVOCs, a three-stage Peltier-based freeze trap 122 was constructed. Freeze trap 122 includes three stacked Peltier chips (e.g., obtained from Ferrotec, Bedford, N.H.) that are cemented to a spiraled Silcosteel® tube of length 30 cm, outside diameter 0.8 mm, and inside diameter 0.53 mm. A heat sink and fan were also incorporated into freeze trap 122. Freeze trap 122 was able to achieve a stable temperature of −30° C. while in operation atop GC unit 118.

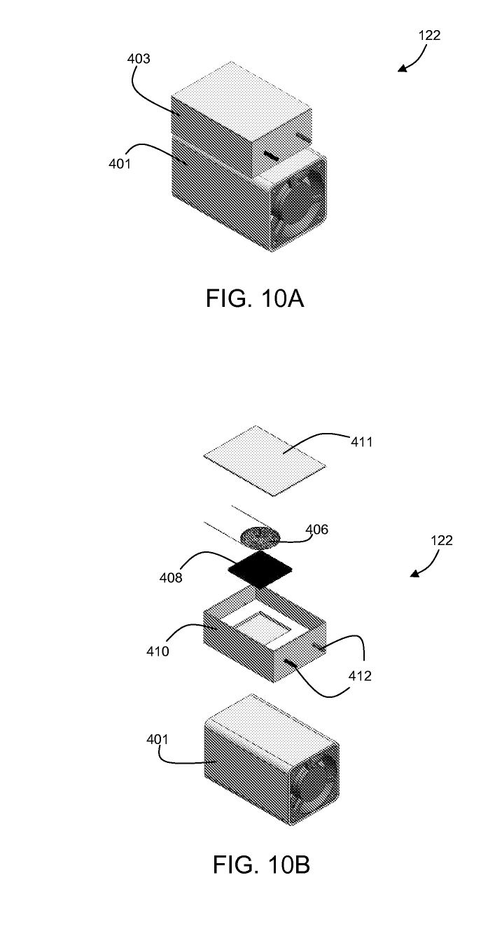

FIGS. 10A and 10B show schematic and exploded views of freeze trap 122, respectively. Freeze trap 122 includes a fan unit 401 and a Peltier-based cooler 403. As shown in FIG. 10B, cooler 403 includes a housing 410 and electrical voltage supply lines 412. Gases—including carrier gas and analytes—flow through condensing tube 406, which is typically formed as a coiled Silcosteel® tube. Organic compounds are trapped via condensation in tube 406. Peltier cooling chip stack 408 controls the temperature of tube 406, and is implemented as a stacked array of three Peltier chips. Coil 406 and chip stack 408 are enclosed by housing 410 and lid 411.

A particular advantage of the three-stage Peltier-based freeze trap 122 is that in some embodiments, freeze trap 122 can also function as thermal desorber 124. During operation, to vaporize trapped VOCs and SVOCs, the Silcosteel® tube in freeze trap 122 can be resistively heated (e.g., by passing an electrical current through the tube under the control of processing unit 128) to a temperature of about 280° C. in less than 10 s. At this temperature, trapped VOCs and SVOCs are vaporized, and are carried by flowing helium gas (e.g., at a flow rate of about 1.0 mL/min and 4.96 kPa) to GC unit 118 and MS unit 120 for characterization.

During operation, processing unit 128 can be configured to control intervals during which organic compounds are condensed in freeze trap 122, and intervals during which the condensed compounds are desorbed from freeze trap 122. In some embodiments, for example, processing unit 128 controls freeze trap 122 so that organic compounds are condensed in the freeze trap (e.g., in the spiraled Silcosteel® tube) for a period of between 3 and 7 minutes (e.g., between 4 and 6 minutes, between 4.5 and 5 minutes). In certain embodiments, processing unit 128 controls freeze trap 122 so that the condensed organic compounds in the freeze trap are heated to cause desorption for a period of between 0.5 and 4 minutes (e.g., between 1 and 3 minutes, between 2 and 2.5 minutes). In general, the interval during which condensation occurs can be larger than the interval during which vaporization occurs by a factor of 1.0 or more (e.g., by a factor of 1.5 or more, by a factor of 2.0 or more, by a factor of 2.5 or more, by a factor of 3.0 or more).

In some embodiments, other freeze trap configurations can also be used in system 100. For example, freeze trap 122 can include, be connected to, or be replaced by a spiraled Silcosteel® tube coupled to a liquid carbon dioxide or liquid nitrogen cryotrap (e.g., freeze trap 307 in FIG. 9A). Typically, the Silcosteel® tube is positioned within a metal sleeve through which cryogenic fluid passes to trap VOCs and SVOCs in the tube. A liquid carbon dioxide cryotrap that achieves an operating temperature of −50° C. has been constructed. In such freeze traps, the interior Silcosteel tube can still be resistively heated, and thus these freeze traps can also function as thermal desorber units, although additional heating time is required to reach temperatures of 280° C. relative to the Peltier-based freeze trap disclosed above.

Although described above in connection with system 100, the Peltier-based freeze trap disclosed herein can more generally be used in a variety of applications where collection, concentration, and desorption of organic compounds occurs. In particular, the design of freeze trap 122, which permits functioning both as a trap and as a thermal desorber, enables low-energy, efficient trapping and concentration of compounds at temperatures of −30° C., even in the presence of significant heat sources. Moreover, resistive heating of the condensing coil within freeze trap 122 achieves efficient desorption without any additional hardware components, making freeze trap 122 a compact device well suited for integration. Alternative applications of freeze trap 122 include, but are not limited to, indoor and/or outdoor air monitoring, process monitoring in manufacturing (e.g., process stream monitoring), mechanical and/or aeronautical exhaust monitoring, and forensic detection (e.g., police and fire investigations).

### Hardware and Software Implementations

The functions, configurations, and steps described herein can be implemented in hardware or in software, or in a combination of both, by processing unit 128, for example. In particular, instructions that implement the functionality disclosed herein can be embodied in computer programs using standard programming techniques following the steps and functions disclosed herein. The programs can be designed to execute on programmable processors or computers, e.g., microcomputers, each including at least one processor, at least one data storage system (including volatile and non-volatile memory and/or storage elements), at least one input device, such as a keyboard or push button array, and at least one output device, such as a CRT, LCD, or printer. Program code is applied to input data to perform the functions described herein. The output information is applied to one or more output devices such as a printer, or a CRT or other monitor, or a web page on a computer monitor with access to a website, e.g., for remote monitoring.

Each program can be implemented in a high level procedural or object oriented programming language to communicate with a computer system. However, the programs can be implemented in assembly or machine language, if desired. In any case, the language can be a compiled or interpreted language.

Each such computer program can be stored on a non-transitory storage medium or device (e.g., ROM or magnetic diskette) readable by a general or special purpose programmable computer, for configuring and operating the computer when the storage medium or device is read by the computer to perform the procedures described herein. The system can also be considered to be implemented as a computer-readable storage medium, configured with a computer program, where the storage medium so configured causes a processor in the computer to operate in a specific and predefined manner to perform the functions described herein. Additional details regarding certain hardware and software features of the systems and methods disclosed herein are described, for example, in U.S. Pat. No. 6,487,920, the entire contents of which are incorporated herein by reference.

### Applications

In addition to site characterization applications disclosed above, the methods and systems disclosed herein have other applications and uses as well. For example, the systems disclosed herein can be used in manufacturing facilities to monitor industrial processes that generate (or may generate) organic by-products. A transfer line (e.g., similar to transfer line 106) with an inlet probe can be positioned in a strategic location along a process line, and the transfer line can collect organic compounds for analysis. By monitoring the emission of such compounds, for example, production rates, safety hazards, and waste generation can be monitored from a remote location.

In some embodiments, the systems and methods disclosed herein can be used on industrial emissions stacks to monitor concentrations of organic waste products in discharged gases. One or more inlet probes can be mounted to the walls of an effluent stack for example; the inlet probes can be connected via one or more transfer lines to an analysis unit at a remote location, which can characterize and measure concentrations of various organic compounds in effluent flows.

In certain embodiments, the systems and methods disclosed herein can be used in “sick” buildings to identify and locate various types of chemical and biological hazards. VOCs that are emitted by building materials such as paints, carpets, wall coverings, and ceiling tiles can be detected using inlet probes mounted on transfer lines. The presence of mold can be detected by monitoring emissions of certain metabolic organic by-products. For example, the following metabolic by-products are produced by various types of mold and/or fungi spores, and can be detected using the systems and methods disclosed herein: butanols, pentanols, hexanols, heptanols, octanols, butenols, pentenols, hexenols, heptenols, octenols, butanones, pentanones, hexanones, heptanones, octanones, sesquiterpenes, heptanes, octanes, nonanes, decanes, undecanes, heptanoic acids, octanoic acids, nonanoic acids, decanoic acids, undecanoic acids, methylpyrazines, and β-Farnesenes. Inlet probes mounted on flexible transfer lines can travel through air ducts and between walls to identify and characterize different types of mold spores in hard-to-reach locations. By identifying and locating such hazards, buildings can be made significantly safer for their occupants.

### EXAMPLES

The following examples present further features and aspects of the systems and methods disclosed herein, but are not intended to limit the scope of the disclosure described in the claims.

To evaluate the system, a series of laboratory and field tests were performed. In the laboratory tests, soil contaminated with fuel oil from an underground storage tank in Massachusetts was collected and analyzed by advancing an inlet probe through the soil by hand. In the field tests, the system was mounted on the back of a truck (see FIG. 2, for example) and transported to the site of a former manufactured gas plant in North Carolina. A cone penetrometer was used to advance the inlet probe and transfer line into the soil. Both soil and groundwater were sampled using the inlet probe.

A Gerstel (Mulheim an der Ruhr, Germany) modular accelerated column heater (MACH) and Agilent (Santa Clara, Calif.) model 5890/5972 GC-MS instrument was modified for field testing. The MACH resistively heated the GC column, providing fast temperature programming and cool down. The Agilent GC oven was used to heat the transfer line from injection port to the GC-MS instrument. For the laboratory tests, a Shimadzu (Columbia, Md.) model 17A/QP5050A GC-MS instrument was used to analyze compounds. Field and laboratory operating conditions are summarized in Table 1. The GC-MS data from both laboratory and field tests were analyzed using the Quantitative Deconvolution software available from Ion Signature Technology (North Smithfield, R.I.). Target compound response factors were calculated as the ratio of AₓCᵢₛ/AᵢₛCₓ, where Cₓ was the amount of target analyte introduced into the GC-MS, Aₓ was its observed signal, and Cᵢₛ and Aᵢₛ were the concentration and observed signal for an internal standard compound. A custom mix of benzene, toluene, ethyl benzene, and xylenes (BTEX), polyaromatic hydrocarbons (PAHs), and internal standards was prepared by Organic Standards Solutions International (Charleston, S.C.). A PIANO aromatics standard solution was obtained from AccuStandard (New Haven, Conn.).

TABLE 1

| | Field Instrument | Laboratory Instrument |
|---|---|---|
| Gas Chromatograph | Agilent 5890 Series II | Shimadzu GC-17A |
| Mode | splitless, 0.4 μL injection | splitless, 1 μL injection |
| Inlet Temperature | 280° C. | 280° C. |
| Pressure | 4.96 kPa | 85.08 kPa |
| Carrier Gas | helium | helium |
| Linear Velocity | 55.5 cm/s | 48.1 cm/s |
| Transfer Line Temperature | 300° C. | 300° C. |
| Column | MACH/Restek Rxi-5Sil MS | Restek Rxi-XLB |
| Length | 15 m | 30 m |
| Diameter | 0.32 mm | 0.25 mm |
| Film Thickness | 0.25 μm | 0.25 μm |
| Mode | constant flow 1.0 mL/min | constant flow 1.8 mL/min |
| Initial GC Conditions | 30° C. | 35° C. |
| | (1.00 min., isothermal) | (2.00 min., isothermal) |
| Ramp 1 | 85° C./min. to 310° C. | 4° C./min. to 55° C. |
| | (0.70 min.) | (0.00 min.) |
| Ramp 2 | none | 6° C./min. to 310° C. |
| | | (6.50 min.) |
| Total Run Time | 4.99 min. | 56.00 min. |
| Mass Spectrometer | Agilent 5972 | Shimadzu GCMS QP5050A |
| Solvent Delay | 0.00 min. | 2.94 min. |
| EM Voltage | 1,671 V | 1,300 V |
| Low Mass | 50 amu | 50 amu |
| High Mass | 285 amu | 350 amu |
| Threshold | 500 | 500 |
| Scan Rate | 5 s⁻¹ | 5 s⁻¹ |

As the inlet probe was advanced through the soil, photoionization signals were measured using PID 130 at different soil depths between 0.61 m and 14.5 m. The background response for each soil sample was determined from photoionization signals measured in the first 5-10 cm of soil depth. These signals were averaged and subtracted from subsequent signals corresponding to greater soil depths. Negative signals for greater soil depths were assumed to indicate that no detectable organic contaminants were present. A positive signal at a greater depth was assumed to correspond to a relative measure of coal tar concentration at that depth. Information about the presence and absence of organic contaminants at particular soil depths could be used to construct a conceptual site model, for example.

Field and laboratory GC-MS data at a soil depth of 8.53±0.30 m are shown in Table 2. Field results were based on n=3 discrete in situ measurements recorded over a depth range of 60 cm. As shown in Table 2, the precision of such measurements (estimated by the percent relative standard deviation, % RSD) was excellent for all analytes. Laboratory data were determined from a composite soil sample collected in a 120 cm tube at the same approximate depth and boring. Sub-samples from the tube were homogenized, extracted, and analyzed according to EPA methods.

TABLE 2

| | | Field | |
|---|---|---|---|
| Compounds | Lab | (% RSD) | % Recovered |
| Benzene | 11 | 9 (24) | 82 |
| C1-Benzene | 328 | 228 (35) | 70 |
| C2-Benzenes | 639 | 559 (6) | 87 |
| Naphthalene | 1,439 | 2,255 (28) | 157 |
| C1-Naphthalenes | 913 | 914 (38) | 100 |
| C2-Naphthalenes | 1033 | 904 (33) | 88 |
| Acenaphthylene | 224 | 173 (18) | 77 |
| Acenaphthene | 144 | 110 (29) | 76 |
| Fluorene | 380 | 170 (27) | 45 |
| Phenanthrene | 640 | 275 (22) | 43 |

Measurement accuracy was acceptable for every compound except for naphthalene (overestimated), fluorene (underestimated), and phenanthrene (underestimated). The under-estimation of higher molecular weight fluorenes and phenanthrenes may be related to limitations on the temperature to which the inlet probe can be heated (e.g., approximately 120° C.), thereby reducing the efficiency with which higher molecular weight organic compounds can be volatilized.

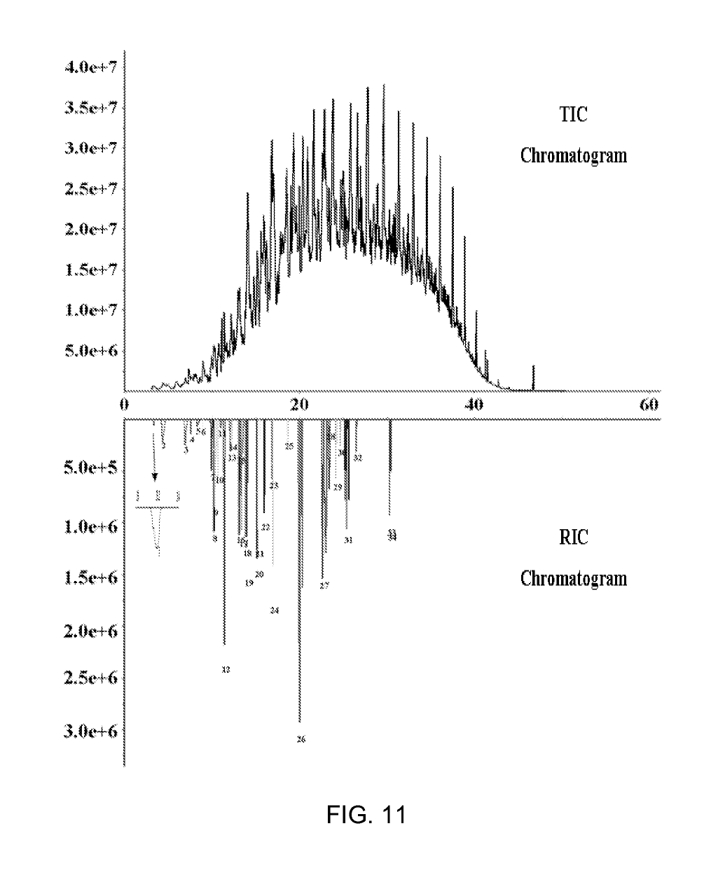

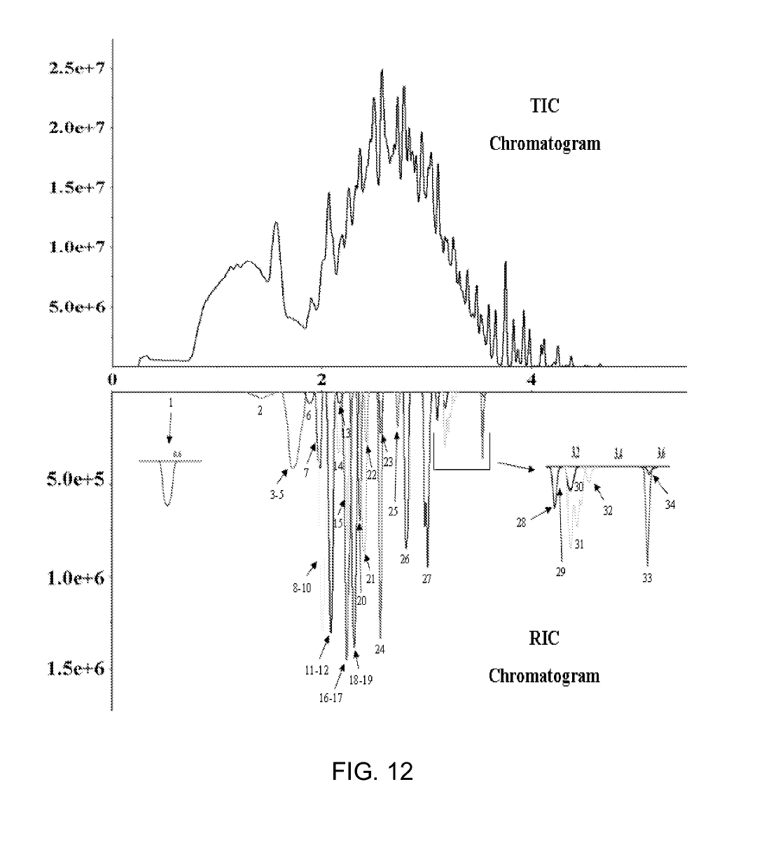

FIGS. 11 and 12 show the total ion current (TIC) and reconstructed ion current (RIC) chromatograms for the field and lab tests, respectively. Tables 3 and 4 identify various peaks in these chromatograms. Absent in the field chromatogram is the well-defined hydrocarbon profile that is present in the laboratory (solvent-extracted) chromatogram.

Although measurement sensitivity is limited by the temperature to which the inlet probe can be heated, reported limits for PAH standards are similar to MIP detection limits. The analysis software deconvolves target compound fragmentation patterns from matrix spectra much more efficiently than standard analysis software, for which reporting limits are often 10-100 times larger than the laboratory method's detection limit. The deconvolution software eliminates sample dilution requirements, and yields data in approximately 5 minutes.

No false positives or negatives were observed when comparing field and laboratory data for VOCs, which is particularly significant considering that benzene, isopropyl benzene, and acenaphthylene are present in concentrations of only a few hundred micrograms per kilogram in the soil. Although the precision of the sensor measurements in the field tests was poorer than the precision of the laboratory tests, differences may be due in part to discrete rather than composite sample collection methods. Nonetheless, the average C₀-C₆ benzene RSD was 26%, well within the EPA's data quality objectives. Although poorer, the 45% RSD for PAHs provides reliable concentration estimates when constructing conceptual models of site contamination. The VOC/SVOC data were within the 50% criterion established by the EPA for field studies.

Based on site-specific action levels outlined in the EPA's Soil Screening Guidance Document, measurement accuracy was also excellent. Applying the most stringent action level—namely, when pollutants are in close proximity to shallow water tables, fractured media, or have source sizes greater than 0.12 km²—the quantitation limit (QL) is set to one-half the action level. To meet the EPA criterion for accuracy, the relative percent difference (RPD) between field and laboratory tests should be less than 60% for target compounds whose concentrations are greater than five times the QL. For concentrations less than five times the QL, the RPD should be less than 100%.

TABLE 3

| | | Lab Avg. | Field Avg. | EPA Ace. | Ace. | |
|---|---|---|---|---|---|---|
| No. | Compounds | (% RSD) | (% RSD) | Criterion | RPD | RPD |
| 1 | Benzene | 0.1 (34) | 0.1 (35) | 0.005 | <60% | 0 |
| | C1-Benzene | 1.6 (13) | 0.9 (7) | 1.5 | <60% | 56 |
| 2 | Toluene | | | | | |
| | C2-Benzenes | 6.5 (10) | 7.8 (16) | | | −18 |
| 3 | ethylbenzene | | | 25 | <60% | |
| 4 | m-, p-xylene | | | 2 | <60% | |
| 5 | o-xylene | | | | | |
| | C3-Benzenes | 26.8 (6) | 21.1 (23) | | | 24 |
| 6 | Isopropylbenzene | 0.5 (7) | 0.4 (23) | N/A | | 22 |
| 7 | n-Propylbenzene | 1.7 (4) | 1.7 (22) | N/A | | 0 |
| 8 | 1-Methyl-3-Ethylbenzene | 11.8 (9) | 10.1 (23) | N/A | | 16 |
| 9 | 1-Methyl-4-Ethylbenzene | | | | | |
| 10 | 1-Methyl-2-Ethylbenzene | | | | | |
| 11 | 1,3,5-Trimethylbenzene | 12.8 (5) | 8.9 (23) | N/A | | 36 |
| 12 | 1,2,4-Trimethylbenzene | | | | | |
| | C4-Benzenes | 31.7 (5) | 23.0 (27) | | | 32 |
| 14 | tert-Butylbenzene | 1.5 (8) | 1.9 (28) | N/A | | −24 |
| 15 | n-Butylbenzene | 3.6 (4) | 3.0 (27) | N/A | | 18 |
| 16 | 1-Methyl-3-n-Propylbenzene | 7.3 (7) | 5.3 (25) | N/A | | 32 |
| 17 | 1-Methyl-4-n-Propylbenzene | | | | | |
| 18 | 1,3-Dimethyl-5-Ethylbenzene | 8.7 (2) | 5.8 (24) | N/A | | 40 |
| 19 | 1,4-Dimethyl-2-Ethylbenzene | | | | | |
| 20 | 1,2-Dimethyl-3-Ethylbenzene | 4.2 (3) | 2.6 (22) | N/A | | 47 |
| 21 | 1,2,4,5-Tetramethylbenzene | 6.4 (4) | 4.4 (34) | N/A | | 37 |
| | C5-Benzenes | 8.6 (3) | 6.0 (28) | N/A | | 36 |
| 22 | n-Pentylbenzene | | | | | |
| | C6-Benzenes | 3.9 (33) | 2.1 (48) | N/A | | 60 |
| 25 | 1,3,5-Triethylbenzene | | | | | |
| | PAH | | | | | |
| 24 | Naphthalene | 4.4 (34) | 3.5 (47) | 10 | <100% | 23 |
| 28 | Acenaphthylene | 0.5 (4) | 0.4 (50) | 70 | <100% | 22 |
| 30 | Acenaphthene | 0.9 (4) | 0.5 (43) | 70 | <100% | 57 |
| 32 | Fluorene | 0.9 (4) | 0.4 (32) | 70 | <100% | 77 |
| 34 | Phenanthrene | 1.1 (2) | 0.1 (50) | 710 | <100% | 167 |

TABLE 4

| No. | Homolog | Lab | Field | % Diff |
|---|---|---|---|---|
| 26 | C1-Naphthalenes | 16.2 | 8.5 | 48 |
| 27 | C2-Naphthalenes | 25.5 | 12.6 | 51 |
| 31 | C3-Naphthalenes | 17.9 | 8.6 | 52 |
| | ΣC1-C3 | 60 | 30 | |

Referring to Table 3, the QLs for benzene, toluene, and C2-benzenes are 0.001, 0.3, and 5 mg kg⁻¹, respectively, and the QL for ethylbenzene is 0.4 mg kg⁻¹. The QLs for naphthalene, acenaphthylene, and acenaphthene are 2, 14, and 14 mg kg⁻¹, respectively. Accordingly, based on the QL values, detection of all of these compounds falls within the EPA's criterion for accuracy. In particular, these results demonstrate that the three-stage Peltier freeze trap used in the system efficiently captured VOCs.

The data in Table 4 shows that fuel oil weathering can be deduced based on the loss of the C0 and C1 analogs compared to the amount of C0-C4 naphthalenes detected. The absence of C4-naphthalenes and the high concentration of C0 compared to total alkylated naphthalenes suggests a relatively new release occurred, which is consistent with the hydrocarbon backbone observed in FIG. 12. Methods for analyzing the data in Tables 1-4 are disclosed, for example, in C. Ziegler et al., “Total alkylated polycyclic aromatic hydrocarbon characterization and quantitative comparison of selected ion monitoring versus full scan gas chromatography/mass spectrometry based on spectral deconvolution,” Journal of Chromatography A 1205(1-2): 109-116 (2008), the entire contents of which are incorporated by reference herein.

These results demonstrate that the on-line detection and characterization by the systems disclosed herein can yield contaminant profiles as the inlet probe is continuously advanced into the subsurface. The combination of the on-line GC-MS analysis unit and the spectral deconvolution software correctly quantifies target compounds in approximately five minutes. No false negatives were observed even at low analyte concentrations, and data quality was consistent with EPA criteria for measurement precision and accuracy. The systems can be used to detect a wide range of organic contaminants in soil and groundwater.

To demonstrate the ability of the inlet membranes disclosed herein to yield results that are comparable to direct syringe injection of calibration standards into a GC/MS analyzer, a membrane was prepared as disclosed herein, and then a stainless steel injection port was sealed against the membrane face using a clamp and a Teflon® o-ring. A series of analytes were injected through a rubber septurm and the injection port into this closed sample introduction system. The injection syringe was nearly touching the membrane face when the analytes were introduced.

The membrane was maintained at a temperature of 300° C. while analytes passed through. A stream of nitrogen swept the analytes through a transfer line heated to 280° C. and into a trap with a three-stage Peltier-based cooler. The trap was maintained at a temperature of −30° C. for a sample collection period of 5 minutes. Thereafter, the flow of carrier gas in the trap was reversed, and the adsorbed analytes were thermally desorbed at a temperature of 260° C. for a period of 2 minutes. The desorbed analytes were transported by the carrier gas to a GC/MS unit for analysis.

Table 5 shows the measured results for a variety of different analytes. In general, the results are consistent with standard syringe injection laboratory GC/MS calibration curves. The data in Table 5 are particularly accurate in view of the dead volume that was present between the rubber septum and the membrane. In typical laboratory analyses, a carrier gas sweeps the organic analytes from the injection port to the GC unit. In field applications, however, no such sweeping occurs, because the soil matrix is typically compacted against the membrane when it is positioned underground. The results in Table 5 demonstrate that even in such conditions, accurate measurement results can be obtained.

TABLE 5

| Compound | Range | RF | % RSD |
|---|---|---|---|
| Naphthalene | 8-750 | 1.09 | 26 |
| Acenaphthylene | 8-1000 | 6.31 | 18 |
| Fluorene | 8-1000 | 0.58 | 21 |
| Phenanthrene | 8-750 | 1.31 | 10 |
| Anthracene | 8-750 | 0.90 | 12 |
| Fluoranthene | 8-750 | 1.05 | 11 |
| Pyrene | 8-750 | 0.78 | 23 |
| Benzo[a]anthracene/Chrysene | 25-1000 | 0.64 | 23 |
| Benzo[b/k]fluoranthene | 25-1000 | 1.46 | 29 |
| Benzo(a)pyrene | 25-1000 | 2.04 | 5 |
| Indeno(1,2,3-c,d)pyrene | 50-1000 | 1.24 | 21 |
| Dibenz(a,h)anthracene | 50-1000 | 1.38 | 44 |
| Benzo(g,h,i)perylene | 50-1000 | 1.40 | 23 |

### Other Embodiments

A number of embodiments have been described. Nevertheless, it will be understood that various modifications may be made without departing from the spirit and scope of the disclosure. Accordingly, other embodiments are within the scope of the following claims.

What is claimed is:

1. A system for detecting organic compounds, the system comprising:
a transfer line;
a probe connected to a first end of the transfer line, the probe comprising an inlet port and a membrane positioned across an opening in the inlet port;
an analysis unit connected to a second end of the transfer line and comprising:
a valve comprising multiple ports, wherein a first one of the multiple ports is connected to the transfer line;
a first detection unit configured to measure a photoionization current for chemical compounds and connected to a second one of the multiple ports;
a second detection unit configured to identify chemical compounds and connected to a third one of the multiple ports;
a trap configured to condense chemical compounds from a vapor phase to a liquid phase, and connected to a fourth and a fifth ones of the multiple ports; and
an electronic controller connected to the valve, the first and second detection units, and the trap.

2. The system of claim 1, wherein the membrane comprises a fluorinated coating material applied to a supporting material, and wherein the membrane does not undergo degradation when heated to a temperature of 300° C.

3. The system of claim 1, wherein a thickness of the fluorinated coating material is 50 microns or less.

4. The system of claim 1, wherein the membrane is impermeable to water and steam, and wherein the membrane is permeable to at least some organic molecules.

5. The system of claim 1, further comprising a first sample injector connected between the second port and the first detection unit.

6. The system of claim 5, further comprising a second sample injector connected to a sixth one of the multiple ports, the second sample injector comprising an aperture configured to admit a syringe.

7. The system of claim 1, wherein the trap comprises:
a condenser coil forming a flow path for gas, and a first set of electrical contacts connected to the condenser coil; and
a cooling element adjacent to the condenser coil, and a second set of electrical contacts connected to the cooling element.

8. The system of claim 7, wherein the cooling element comprises a stack of three Peltier cooling chips.

9. The system of claim 7, wherein during operation the cooling element is configured to maintain the condenser coil at a temperature of −30° C. or less.

10. The system of claim 7, wherein the electronic controller is configured to reduce a temperature of the condenser coil by applying an electrical signal to the second set of electrical contacts, and to increase the temperature of the condenser coil by applying an electrical signal to the first set of electrical contacts.

11. The system of claim 1, wherein the probe comprises a heating element connected to the electronic controller, and wherein during operation the electronic controller is configured to apply an electrical signal to the heating element to maintain the probe at a temperature of 300° C. or more.

12. The system of claim 1, wherein the valve comprises a first configuration that defines a first flow path between the first and second ports in the valve, and wherein during operation, the electronic controller is configured to adjust the analysis unit so that the valve is in the first configuration and molecules in the transfer line enter the first port and are detected by the first detector.

13. The system of claim 12, wherein the valve comprises a second configuration that defines a second flow path between the first and fifth ports in the valve, and wherein during operation, when molecules are detected by the first detector, the electronic controller is configured to adjust the analysis unit so that the valve is in the second configuration and molecules from the transfer line are condensed in the trap.

14. The system of claim 13, wherein the first configuration defines a third flow path between the third and fifth ports, and wherein during operation, when the molecules have been condensed in the trap, the electronic controller is configured to adjust the analysis unit so that the valve is in the first configuration and the condensed molecules are detected by the second detector.

15. The system of claim 13, wherein gas flow through the trap occurs in a first direction when the valve is in the first configuration, and in a second direction opposite to the first direction when the valve is in the second configuration.

16. The system of claim 1, wherein a length of the transfer line is 3 meters or more.

17. A method for detecting organic compounds, the method comprising:
directing molecules of one or more organic compounds to flow through a transfer line and to enter a valve through a first one of multiple valve ports, wherein the valve comprises a first configuration defining a flow path between the first port and a second one of the multiple ports;
detecting molecules from the transfer line with a first detector connected to the second port;
adjusting the valve to a second configuration defining a flow path between the first port and a third one of the multiple ports;
condensing molecules from the transfer line in a trap connected to the third port;
adjusting the valve to the first configuration, wherein the first configuration defines a flow path between the third port and a fourth one of the multiple ports;
vaporizing the condensed molecules; and
detecting the vaporized molecules with a second detector connected to the fourth port.

18. The method of claim 17, wherein:
a first end of the transfer line is positioned below a ground surface;
directing molecules of one or more organic compounds to flow through the transfer line comprises thermally desorbing the molecules from a soil matrix material adjacent to the first end of the transfer line; and
thermally desorbing the molecules comprises heating the soil matrix material to a temperature of 300° C. or more.

19. The method of claim 18, wherein the one or more organic compounds comprise at least one volatile organic compound (VOC) and at least one semi-volatile organic compound (SVOC), and wherein the at least one VOC and the at least one SVOC are thermally desorbed at a common temperature from the soil matrix material.

20. The method of claim 18, wherein directing molecules of one or more organic compounds to flow through the transfer line comprises directing the molecules to pass through a membrane comprising a fluorinated coating material that does not degrade at a temperature of 300° C.

21. The method of claim 20, wherein the soil matrix material has a water concentration of 15% or more by weight.

22. A trap for condensing organic compounds, the trap comprising:
a condenser coil forming a flow path for gas;
a cooling element;
a first set of electrical contacts connected to the condenser coil; and
a second set of electrical contacts connected to the cooling element,
wherein during operation the trap is configured to be heated by directing an electrical current to pass through the condenser coil, and configured to be cooled by directing an electrical current to pass through the cooling element.

23. The trap of claim 22, wherein the cooling element comprises a stack of three Peltier cooling chips.

24. The trap of claim 22, further comprising a second cooling element, wherein the condenser coil is positioned between the two cooling elements.

25. A condenser system, comprising:
the trap of claim 22; and
an electronic processor connected to the second set of electrical contacts and configured to condense organic compounds in the condenser coil by directing an electrical current to pass through the cooling element to reduce a temperature of the condenser coil.

26. The condenser system of claim 25, wherein the electronic processor is connected to the first set of electrical contacts and configured to vaporize condensed organic compounds in the condenser coil by directing an electrical current to pass through the condenser coil to increase a temperature of the condenser coil.
